(19) United States
(12) Patent Application Publication
(10) Pub. No.: US 2021/0054434 A1
(43) Pub. Date: Feb. 25, 2021

(54) KIT FOR AMPLIFYING IMMUNOGLOBULIN SEQUENCES

(71) Applicants: Genome Research Limited, Cambridgeshire (GB); Cambridge Enterprise Limited, Cambridgeshire (GB)

(72) Inventors: Velislava Nikolaeva PETROVA, Cambridgeshire (GB); Rachael BASHFORD-ROGERS, Cambridgeshire (GB); Kenneth SMITH, Cambridgeshire (GB); Paul KELLAM, Cambridgeshire (GB)

(21) Appl. No.: 16/317,535

(22) PCT Filed: Jul. 13, 2017
(86) PCT No.: PCT/GB2017/052062

§ 371 Date: Jan. 11, 2019

Related U.S. Application Data
(60) Provisional application No. 62/361,987, filed on Jul. 13, 2016.

(30) Foreign Application Priority Data
Jul. 14, 2016 (GB) 1612242.6

(51) Int. Cl. C12Q 1/686; C12Q 1/6869; G01N 33/53; C07K 16/00

(52) U.S. Cl. CPC C12Q 1/686; C12Q 1/6869; C12Q 2600/118; C07K 16/00; G01N 33/5308

(57) ABSTRACT

The invention relates to a kit for amplifying immunoglobulin sequences and methods thereof, and their use and application in methods for the characterisation of a B-cell repertoire.

### FIELD OF THE INVENTION

The invention relates to a kit for amplifying immunoglobulin sequences and methods thereof, and their use and application in methods for the characterisation of a B-cell repertoire.

### BACKGROUND OF THE INVENTION

Single-cell genetic and transcriptional diversity defines the adaptive immune response, where the combination of the B-cell receptor (BCR) specificity and immunoglobulin isotype contribute to B-cell function and antibody responsivity. BCR genetic diversity is generated through the process of BCR Variable (V), Diversity (D) and Joining (J) gene rearrangement with the addition of non-templated nucleotides for the Ig heavy (IgH) chain and VJ rearrangement for the Ig light (IgL) chain, followed by antigen-driven diversification by somatic hypermutation (SHM) and Ig class-switch. The combination of IgHV and L V(D)J genes encode the variable domains of BCR molecules and confers antigen specificity, while Ig constant genes determine Ig isotypes underlying antibody effector functions. Despite the earlier notion of a bipartite model of antibodies (Abs) with independent variable (Fab) and constant region (Fc) portions, increasing numbers of studies report a more complex relationship between class-switching and antigen specificity (Cooper, L. J., et al. (1993) J Immunol 150, 2231-2242; Dam, T. K., et al. (2008) J Biol Chem 283, 31366-31370; McLean, G. R., et al. (2002) J Immunol 169, 1379-1386), where Ab isotype can affect Ab neutralization (Tudor, D., et al. (2012) Proceedings of the National Academy of Sciences of the United States of America 109, 12680-12685), autoreactivity (Torres, M., et al. (2007) J Biol Chem 282, 13917-13927), and antigen binding affinity (Janda, A., et al. (2012) J Biol Chem 287, 35409-35417; Dodev, T. S., et al. (2015) Allergy 70, 720-724). Furthermore, the specific combination of Ab isotypes can play a synergistic role in B-cell response (e.g. in neutralisation of HIV cell-to-cell transfer (Tudor, D., et al. (2012) supra). This highlights the need for simultaneous assessment of Ab specificity and Ig isotype to build greater insight into the mechanism of co-dependence between SHM and class-switching. Understanding the relationship between these two processes is essential for the accurate characterisation of B-cell responses in health and disease.

Specific Ig isotypes can confer distinct patterns of antibody involvement in immune-mediated diseases and thus may aid the early prediction of autoimmunity (Blanco, F., et al. (1992) Lupus 1, 391-399; van Schaik, F. D., et al. (2013) Gut 62, 683-688) and immune-deficiencies (Peron, S., et al. (2008) The Journal of experimental medicine 205, 2465-2472; Roskin, K. M., et al. (2015) Science translational medicine 7, 302ra135); reveal the mechanism of immune pathology (Verpoort, K. N., et al. (2006) Arthritis Rheum 54, 3799-3808; Bos, W. H., et al. (2008) Annals of the Rheumatic Diseases 67, 1642; Engelmann, R., et al. (2008) Rheumatology (Oxford) 47, 1489-1492) or determine the prognosis of disease progression (Villalta, D., et al. (2013) PloS one 8, e71458). In the context of infectious diseases, the spectrum of Ig isotypes involved in response to a pathogen can highlight inter-host differences in adaptive response (Sanders, L. A., et al. (1995) Pediatr Res 37, 812-819), show specific characteristics of natural vs. vaccine acquired immunity (Nelson, K. M., et al. (1998) Vaccine 16, 1306-1313), reveal the immunogenicity of different vaccine compositions (Visciano, M. L., et al. (2012) J Transl Med 10, 4). This can aid the prediction of vaccine efficacy and guide clinical study progression. In addition to the isotype signatures as a distinctive feature of disease-specific immune responses, preference for certain V genes have also been reported (Foreman, A. L., et al. (2007) Autoimmun Rev 6, 387-401). In multiple sclerosis, a distinctive pattern of SHM and preferential VH4 gene usage are associated with cerebrospinal fluid (CSF) and central nervous system (CNS) response (Owens, G. P., et al. (1998) Ann Neurol 43, 236-243) and have been proposed as a diagnostic tool (Cameron, E. M., et al. (2009) J Neuroimmunol 213, 123-130).

B-cell receptor sequencing provides an opportunity for understanding of B-cell responses in health and disease by characterisation of the genetic basis of antigen-specificity and antibody effector functions. Sequence profiling of Ig repertoires has been applied to the characterization of immune response in infection, vaccination, autoimmunity, and cancer (Francica, J. R., et al. (2015) Nature communications 6, 6565; Rene, C., et al. (2014) Journal of Cellular and Molecular Medicine 18, 979-990; Tan, Y. C., et al. (2014) Arthritis & Rheumatology 66, 2706-2715; Wang, C., et al. (2015) Proceedings of the National Academy of Sciences of the United States of America 112, 500-505). However, to date, there has been no method to capture every isotype class and subclass simultaneously with the BCR sequence. Advances in sequencing along with novel molecular barcoding could enable such techniques to have the potential to replace conventional serological methods for characterisation of B-cell responses in the diagnostic and clinical setting. Recent studies demonstrate utility of immune repertoire sequencing for identification of graft versus host disease post-transplantation (Vollmers, C., et al. (2015) PLoS medicine 12, e1001890); for monitoring immune dynamics during antiretroviral therapy (Hoehn, K. B., et al. (2015) Philosophical transactions of the Royal Society of London. Series B, Biological sciences 370), for identification of disease etiology in multiple sclerosis (Palanichamy, A., et al. (2014) Science Translational Medicine 6, 248ra106). The application of immune repertoire analysis for diagnosis and clinical monitoring of disease requires robust and highly accurate profiling of both antigen-specificity and Ig isotypes. Capturing accurately the full genetic complexity of immune receptor repertoire poses substantial technical challenges and requires careful choice of BCR amplification strategy to ensure the accuracy, sensitivity and fidelity of the amplification and sequencing process. Molecular barcoding allows for correction of PCR and sequencing errors and improves the quantitative potential of immune repertoire analysis. Several strategies for barcode incorporation have been previously described: via template-switching in 5′RACE amplification (Islam, S., et al. (2014) Nature methods 11, 163-166; Shugay, M., et al. (2014) Nature methods 11, 653-655), via barcoded gene-specific primers and nested PCR (Vollmers, C., et al. (2013) Proceedings of the National Academy of Sciences of the United States of America 110, 13463-13468), or during randomly primed cDNA synthesis (Shiroguchi, K., et al. (2012) Proceedings of the National Academy of Sciences of the United States of America 109, 1347-1352).

There is therefore a need to provide improved methods to deconvolute variable gene diversity with isotype class and subclass assignment.

### SUMMARY OF THE INVENTION

According to a first aspect of the invention, there is provided a kit for amplifying immunoglobulin sequences comprising:
(a) two or more first nucleic acid sequences, each of which comprises a 3′ primer which anneals to at least a portion of the constant region of an immunoglobulin class and/or subclass; and
(b) one or more second nucleic acid sequence comprising:
   (i) a 5′ primer comprising a sequence which anneals to at least a portion of each immunoglobulin heavy chain variable gene; or
   (ii) a 5′ template-switching sequence,

wherein when the second nucleic acid sequence is as defined in (b) (ii), the kit additionally comprises a third nucleic acid sequence which is a 5′ primer corresponding to said template-switching sequence.

According to a second aspect of the invention, there is provided a method for amplifying immunoglobulin sequences comprising performing an amplification reaction on cDNA from a biological sample obtained from a human or animal subject, using the kit as defined herein to amplify the immunoglobulin sequences between the first and second nucleic acid sequences.

According to a further aspect of the invention, there is provided a method for characterisation of a B-cell repertoire comprising the method for amplifying immunoglobulin sequences as defined in claim 10, additionally comprising the steps of:
(a) sequencing the amplified product as defined in claim 10 to generate sequencing data; and
(b) computational analysis of the sequencing data in step (a) to characterise the B-cell repertoire.

According to a further aspect of the invention, there is provided a method of computational analysis of the constant and variable regions of an immune receptor, comprising the steps of:
(i) identification of one of said regions of the immune receptor;
(ii) trimming the region identified in step (i) to include the other region of the immune receptor not identified in step (i); and
(iii) joint analysis of both of the regions.

### BRIEF DESCRIPTION OF THE FIGURES

FIG. 1: Comparison of barcode amplification methods.
a) 3′Multiplex PCR (3′MPLX) method; 15nt barcode (5′NNNNTNNNNTNNNNT3′; SEQ ID NO: 1) introduced during reverse transcription (RT) on the reverse J-gene or Constant region (C) primer; forward V gene mix includes 6 primers for Framework Region 1 (FR1); amplicon size: 400 bp.
b) 5′ Multiplex PCR (5′MPLX) method with barcode introduced on each of the 6 V gene primers during the first PCR step; Amplicon size: 400-450 bp.
c) 5′RACE method with a barcode introduced via a template-switch during RT; polyT primer is used for cDNA priming, J non-barcoded primer—for the PCR step; amplicon size: 550 bp.

FIG. 2: Read processing and comparison of primer barcoding methods.

FIG. 3: Sensitivity, reproducibility and barcode profiles.
a) Total counts of VJ gene combinations across the three amplification methods for PBMC sample H1 and LCL1 samples; For sample H1, the mean VJ gene counts across replicates were used.
b) Pearson correlation of VJ gene frequencies across sequenced replicates of H1 sample.
c) Barcode profiles across methods represented as: (i) maximum barcode multiplicity and (ii) % mismatches from consensus in barcode groups; ‘Multiplicity’ was defined as number of BCR reads associated with a unique barcode.
d) Principle Component Analysis of network parameters (a) Vertex Gini Index; b) Cluster Gini index; c) Largest cluster size; d) Second largest cluster size) derived from the captured repertoires of samples H1 in ‘PBMC’ panel and samples LCL1 in ICU panel. Color legend: 3′MPLX-red; 5′MPLX-green; 5′RACE-blue; **** denotes p-value lower than 0.0001, while * shows p-value of lower than 0.03.

FIG. 4: Complete Ig isotype deconvolution of a bulk PBMC sample using IsoTyper.
a) RNA from bulk H2 PBMC samples, amplified with 3′MPLX method and sequenced on IIlumina 300 PE MiSeq Platform. Sequencing data is processed via IsoTyper bioinformatics platform and diversity of Ig repertoire is determined using network analysis (Bashford-Rogers, R. J., et al. (2013) Genome Research 23, 1874-1884). Individual contribution of Ig classes and subclasses to total repertoire diversity is shown with differently colored clones layered on the same network. The blue nodes in the total IgH repertoire can be split into sub-repertoires, where the BCR nodes represented by each immunoglobulin isotype are layered on the same network (red, yellow, green, blue and purple nodes for BCR vertices present in IgHA, IgHD, IgHE, IgHG and IgHM respectively), where the grey nodes represent BCRs in the total repertoire but not represented by the corresponding isotype. In addition, the separate IgHA1-2 and IgHG1-4 repertoires are shown in a similar manner.
b) Evolution of the major network cluster of H2 PBMC repertoire with contribution of individual subclasses to the total cluster phylogeny. The maximum parsimony phylogenetic tree represents the estimate evolutionary relationships between each BCR (nodes), where the nodes are represented by pie-charts corresponding to the proportion of each immunoglobulin isotype observed for each BCR sequence.

FIG. 5: Ig isotype deconvolution of single-cell samples using IsoTyper pipeline.
a) Workflow of single-cell sample processing using IsoTyper platform. HT=High-throughput.
b) Detection of dual expression of IgM/IgD isotypes in singe-cell sample HSC.
c) Expression of a single IgM isotype in single-cell sample H5C7 with the constant region aligned to the reference constant regions.

FIG. 6: IsoTyper characterisation of sorted isotype-specific B-cell populations.

Percentages of each cell-sorted B-cell population represented by corresponding isotype after IsoTyper amplification with a mixture of all isotype-specific primers.

FIG. 7: Step-wise evolution of B-cell populations from naïve to antigen experienced.
a) Schematic of B-cell evolution following antigen stimulation and utility of IsoTyper for detection the BCR diversity each evolutionary stage.
b) The basic structure of a human antibody. The basic structural units of all immunoglobulins are very similar, consisting of two identical heavy chain (IgH) and two identical light (IgL) chain proteins, linked by disulphide bridges. The sites at the tip of the antigen-binding regions are highly diversified and formed from the variable domains of the heavy and light chains, both generated during B-cell development by highly regulated gene rearrangements in the B-cell receptor gene loci. The trunk of the heavy chain protein is known as the constant region, and is defined by the antibody isotype. Although the different isotypes of immunoglobulin have distinct biological activities, structures and distributions throughout the body, and trigger different effector mechanisms, all isotypes of immunoglobulin (IgA, IgD, IgE, IgG, and IgM) can be expressed as a membrane-associated form on the surface of the B-cell (B-cell receptor) or as a secreted form (antibody).
c) Percentages of BCRs of each immunoglobulin isotype class as a percentage of total BCR repertoire (top) and vertex Gini Index (bottom) for each isotype for the healthy individuals (n=19).
d) The percentages of BCRs of each immunoglobulin isotype class exhibiting zero mutations from germline between each isotype subgroup for a healthy individual.
e) (i) Boxplots of the mean number of mutations in clusters exhibiting 2 isotype classes or greater than 2 isotype classes that are either IgM⁺IgD⁺ or IgM⁻IgD⁻ and (ii) boxplots of the cluster sizes of all clusters exhibiting 2 isotype classes or greater than 2 isotype classes that are either IgM⁺IgD⁺ or IgM⁻IgD⁻.
f) Boxplot of correlation coefficients (R²-values) between the naïve BCR (IgM⁺IgD⁺ unmutated) repertoire IgHV-J gene usages and that of each isotype combination. * denotes p-values <0.05, ** denotes p-values <0.005, *** denotes p-values <0.0005 and **** denotes p-values <0.00005.

FIG. 8: IsoTyper sample filtering information for bulk PBMC samples.

FIG. 9: Isotype—specific mutational frequencies in healthy repertoires.
a) Mean numbers of mutations per healthy individual (n=19) per individual immunoglobulin isotype class.
b) Mean numbers of mutations per healthy individual (n=19) per individual immunoglobulin subclass groups.

FIG. 10: Clonal evolution and isotype-restriction of VJ gene usage in healthy repertoires.
a) Maximum parsimony trees showing clonal evolution of three BCR clones from healthy repertoires with simultaneous detection of SHM and class-switching; overlaid pie charts represent total isotype composition of the clone after first class-switch event; in aiii) the phylogenetic tree is shown together with a schematic of the predicted process of B-cell evolution represented by the tree.
b) Differences in V gene family usages between different isotype classes in healthy individuals.
c) Differences in J gene family usages between different isotype classes in healthy individuals.
d) Hierarchical clustering of IgHV-J gene usage frequencies between different class isotypes for healthy individuals. The healthy individual ID denoted by the number in the squared brackets. The P-value of co-clustering between isotype classes was <10⁻¹⁰ (as calculated from Wlcoxon test between the inter-isotype class distances compared to the intra-isotype class distances).

FIG. 11: IsoTyper analysis of B-cell diversity of CLL repertoire.
a) Percentages of BCRs of each immunoglobulin isotype class as a percentage of total BCR repertoire (top) and vertex Gini Index (bottom) for each isotype for the CLL patients (n=6).
b) Bar chart of the percentages of isotype class usages of the CLL cluster for each CLL patient (top) (square root scale used), and heatmap of the isotype class usage of the CLL cluster per CLL patient sample (white to red scale corresponds to low to high proportions of the clone). The CLL samples were hierarchically clustered according to isotype usage frequency similarity (left).
c) The mean number of mutations away from the central BCR in the CLL clone for each isotype class for each patient (time 0 samples only).
d) Joint probability networks between BCRs sharing isotype class types for (i) healthy individual and (ii) CLL patient samples. The node sizes represent the total numbers of unique BCRs represented by the corresponding isotype, and the edge strengths (edge widths and labels) correspond to the joint class isotype probabilities, averaged over the patients in each group.
e) Evolution of the leukemic clusters of CLL patient 2, with contribution of individual subclasses to the total cluster phylogeny. The maximum parsimony phylogenetic tree represents the estimate evolutionary relationships between each BCR (nodes), where the nodes are represented by pie-charts corresponding to the proportion of each immunoglobulin isotype observed for each BCR sequence.

FIG. 12: Frequency and diversity of isotype classes in healthy and in CLL repertoires.

Percentages of BCRs of each immunoglobulin isotype class as a percentage of total BCR repertoire (top) and vertex Gini Index (bottom) for each isotype between healthy individuals (n=19) and CLL (n=6) samples (time 0, PBMCs only). ** denotes p-values <0.05 and *** denotes p-values <0.005.

FIG. 13: Isotype overlap probabilities in healthy and in CLL repertoires

Boxplots of the statistically different overlap probabilities between BCRs sharing isotype class types for healthy individuals (red) and CLL patient samples (green). ** denotes p-values <0.05 and *** denotes p-values <0.005.

FIG. 14: Isotype-specific mutational frequencies in healthy repertoires.
a) Mean numbers of somatic mutations per healthy individual (n=19) per immunoglobulin isotype class in total peripheral blood.
b) Mean numbers of somatic mutations per healthy individual (n=29) per immunoglobulin isotype class in cell-sorted B-cell populations. * denotes p-values <0.05, ** denotes p-values <0.005, *** denotes p-values <0.0005 and **** denotes p-values <0.00005.

FIG. 15: BCR sequencing for clone tracking in B-lymphoblastic leukaemia and monitoring disease.
a) qPCR target/control (T/C) transcript ratios (blue) and percentages of RNA-derived clonotypic B-ALL BCR reads over time for each patient (red for largest cluster and green for second largest cluster, where present). The blue axes (right of each plot) refer to the T/C qPCR transcript ratios levels and the red axes (left) to the percentage of sequences in the corresponding clusters (log 2 scales). Blue and red bars under each plot indicate time-points that are positive for qPCR transcripts and B-ALL BCR reads respectively. The initial sample for patient 1703 was taken 2 weeks after starting treatment, hence the low levels of qPCR and clonotypic BCR positivity at time 0. BM=bone-marrow, PB=peripheral blood and CSF=cerebrospinal fluid sample.
b-c) RNA from a B-ALL patient sample was mixed with RNA from healthy peripheral blood PBMCs at different ratios. BCR sequencing was performed using the full set of multiplex primers or the single primer with the best alignment to the malignant B-ALL BCR sequence (IgHV specific primer), each yielding an average of 125,642 filtered BCR sequences (range of 18,970-294,354). b) Network diagrams showing sequential dilution of B-ALL into healthy blood RNA using the multiplex primers, where clusters within 8 bp sequence similarity to the B-ALL cluster are marked in red and all others in blue. c) Percentages of BCR sequences corresponding to the B-ALL BCR population at each dilution using multiplex primers (dark-green) and IgHV specific primer (dark-red). Overlaid are the percentage in the largest BCR cluster (irrespective of relationship to B-ALL) for multiplex primers (light-green) and IgHV specific primer (light-red).

FIG. 16: Detecting and monitoring secondary IgHV rearrangements in B-lymphoblastic leukaemia subclones.
a) Schematic representation of different mechanisms of secondary IgHV rearrangements. i) Independent IgHV rearrangements: After the D-J rearrangement, an early B cell divides and the resulting cells undergo independent IgHV rearrangements, whilst retaining a common IgHD-J stem sequence. ii) IgHV replacement: an upstream IgHV gene is rearranged onto a pre-existing D-J rearrangement.
b) High-throughput detection of secondary rearrangements in B-ALL patient samples for (i) patient 859, (ii) patient E and (iii) patient F. The percentages of BCR sequences containing the stem sequences from the major clones in each patient were identified in serial time points (encompassing the IgHD-IgHJ region and non-template additions up to 3 bp 3′ to the end of the IgHV gene). Different IgHV gene usages are plotted in different colours, and the highest three observed IgHV genes indicated above the plots. The grey lines indicate the top 99^{th} percentile frequency of each stem sequence in 18 healthy individuals (0% for (i)-(iii)).
c) Network diagram for B-ALL patient 859 at day 0, with vertices within the largest cluster (Cluster 1) in red, vertices within the second largest cluster (Cluster 2) in green and all other vertices in blue. d) BCR sequence alignment of the dominant sequences from the two dominant clusters in patient 859, cluster 1 and cluster 2 representing 2.81% and 2.89% of BCRs respectively. The cluster 1 and 2 sequences were aligned to each other, and the positions of differences between sequences are indicated by the coloured boxes in the corresponding positions in the middle row, using red for mismatches, green for gaps in cluster 1 BCR and blue for gaps in cluster 2 BCR. The cluster 1 and 2 sequences were 100% identical to the germline genes of [IgHV4-34-IgHD4-11-IgHJ6] and [IgHV1-2-IgHD4-11-IgHJ6] respectively, where the red, blue and green boxes for IgHV, D and J genes mark the gene boundaries respectively.
e-g) Alignments of the two largest BCR sequence clusters for patient 859 (e), patient E (f) and patient F (g). The alignments with the reference IgHV (highlighted in red), IgHD (highlighted in yellow) and IgHJ (highlighted in green) genes are indicated with dashes (-) denoting alignment gaps. The regions of the BCR sequence that are identical between the two clusters are highlighted in the grey boxes.

FIG. 17. A maximum parsimony phylogenetic tree of a representative IgE-associated clonal expansion in an EGPA patient at diagnosis (0 months).

Colours correspond to the isotype usage for each BCR. All nodes are scaled to unitary size.

### DETAILED DESCRIPTION OF THE INVENTION

Kit

According to a first aspect of the invention, there is provided a kit for amplifying immunoglobulin sequences comprising:
(a) two or more first nucleic acid sequences, each of which comprises a 3′ primer which anneals to at least a portion of the constant region of an immunoglobulin class and/or subclass; and
(b) one or more second nucleic acid sequence comprising:
   (i) a 5′ primer comprising a sequence which anneals to at least a portion of each immunoglobulin heavy chain variable gene; or
   (ii) a 5′ template-switching sequence,

wherein when the second nucleic acid sequence is as defined in (b) (ii), the kit additionally comprises a third nucleic acid sequence which is a 5′ primer corresponding to said template-switching sequence.

Disclosed herein, IsoTyper, is the first strategy to date for complete deconvolution of variable gene diversity with isotype class and subclass assignment in a single reaction, allowing for the functional characterisation of B-cell responses in health and disease. IsoTyper is based on a carefully optimised methodological framework for barcoded BCR sequencing to minimise technical noise and to enable accurate biological inferences. IsoTyper has been used to demonstrate a higher degree of complexity of the immune architecture in health with isotype-restriction of variable gene usage and distinct patterns of clonal evolution of individual Ig subtypes. In addition, class-switch recombination (CSR) and isotype-specific evolution of pathological clones in the context of disease, which is undetected on the variable gene sequence level, has also been shown. This highlights the unique enabling utility of IsoTyper to detect subtle changes in B-cell responses and thus contribute to the understanding of disease progression.

The kit defined herein advantageously allows for the parallel amplification of all immunoglobulin classes and subclasses in a single PCR reaction. This enabled capture of both immunoglobulin heavy chain (IgH) VDJ and constant region genes providing high-resolution repertoire characterization from a single biological sample. In addition, multiplex BCR amplification with primer barcoding during reverse transcription (3′MPLX) was shown to be the most efficient at detecting immune repertoire diversity capturing between 9-90× more unique RNA molecules, with increased sensitivity of transcript recapture for low frequency BCRs.

References to the term “immunoglobulin” as used herein refer to a protein which is produced by the B-cells of the immune system, in particular plasma cells, in response to bacteria, viruses, fungus, allergens, cancer cells or host cells. Immunoglobulins are also known as antibodies and the molecules they recognise are known as antigens. Antibodies can occur in a soluble form, that is secreted from the cell to be free in the blood plasma, and a membrane-bound form, that is attached to the surface of a B-cell and is referred to as the B-cell receptor (BCR).

Structurally, antibodies are glycoproteins that typically comprise basic structural units, each with two large heavy chains and two small light chains. In humans, there are two light chains (κ and λ) and several different types of heavy chains, based on five different types of crystallizable fragments (Fc) that may be attached to the antigen-binding fragments. The five different types of Fc regions allow antibodies to be grouped into five isotypes or classes (α, δ, ε, γ, and μ). Generally, each Fc region of a particular antibody isotype is able to bind to its specific Fc Receptor, thus allowing the antigen-antibody complex to mediate different roles depending on which FcR it binds. Therefore, references to the term “immunoglobulin sequences” as used herein, refer to the nucleic acid sequence (such as a DNA or RNA sequence) of an immunoglobulin.

Common antibody isotypes, also known as classes, include but are not limited to IgG, IgA, IgM, IgE and IgD in placental mammals. Some of these classes may then also be divided into sub-classes, such as IgG (IgG1, IgG2, IgG3 and IgG4) and IgA (IgA1 and IgA2). It will be appreciated by one skilled in the art that the invention disclosed herein also has application in non-mammal species, where immunoglobulins include, but are not limited to: IgF and IgX in Amphibia; IgT and IgZ in bony fish; IgW in cartilaginous fish and lungfish; IgY in Amphibia, reptiles, and birds; IgNAR in sharks; and other non-conventional constant regions in camelid antibodies which exclude the CH1 region in IgG2 and IgG3. Therefore, in one embodiment, the immunoglobulin class and/or subclass is selected from IgA1, IgA2, IgD, IgE, IgG1, IgG2, IgG3, IgG4, IgM, IgK, IgL, IgF, IgT, IgX, IgW, IgY and IgZ IgNAR. In a further embodiment, the immunoglobulin class and/or subclass is selected from IgA1, IgA2, IgD, IgE, IgG1, IgG2, IgG3, IgG4 and IgM.

References to the term “primer” as used herein, refer to a short nucleic acid sequence that serves as a starting point for nucleic acid synthesis. A primer, when used in artificial nucleic acid replication, is often synthetic and often used as part of a pair of primers, 5′ and 3′ (forward and reverse, respectively), which direct replication towards each other. A primer or primers may also be used in nucleic acid sequencing methods. Methods of primer design are widely known in the art.

References to the term “template-switching sequence” as used herein, refer to a nucleic acid sequence designed with at least three consecutive guanine nucleic acids at the 3′ end and a region of known sequence at the 5′ end. It would be known to one skilled in the art that the use of a reverse transcriptase achieves an addition of this template-switching sequence, such as a 5′-RACE linker sequence, due to terminal transferase activity of reverse transcription.

In one embodiment, the kit comprises two or more, three or more, four or more or five or more first nucleic acid sequences. In a further embodiment, the kit comprises five first nucleic acid sequences.

In one embodiment, the 3′ primer anneals to at least a portion of the constant region of IgA (IgA1 and IgA2) and comprises the sequence: GAYGACCACGTTCCCATCT (SEQ ID NO: 2).

In an alternative embodiment, the 3′ primer anneals to at least a portion of the constant region of IgM and comprises the sequence: TCGTATCCGACGGGGAATTC (SEQ ID NO: 3).

In an alternative embodiment, the 3′ primer anneals to at least a portion of the constant region of IgD and comprises the sequence: GGGCTGTTATCCTTTGGGTG (SEQ ID NO: 4).

In an alternative embodiment, the 3′ primer anneals to at least a portion of the constant region of IgE and comprises the sequence: AGAGTCACGGAGGTGGCATT (SEQ ID NO: 5).

In an alternative embodiment, the 3′ primer anneals to at least a portion of the constant region of IgG (IgG1, IgG2, IgG3 and IgG4) and comprises the sequence:

(SEQ ID NO: 6)
AGTAGTCCTTGACCAGGCAG.

In one embodiment, when the second nucleic acid is as defined in step (b) (i), the two or more first nucleic acid sequences each additionally comprise a detectable label.

In one embodiment, the two or more first nucleic acid sequences each additionally comprise a non-annealing nucleic acid sequence, which is identical in each of said two or more first nucleic acid sequences, and the kit additionally comprises a third nucleic acid sequence complementary to said non-annealing nucleic acid sequence. Therefore, in one embodiment, the 3′ primer anneals to at least a portion of the constant region of IgA (IgA1 and IgA2) and comprises the sequence:

(SEQ ID NO: 7)
TGTCCAGCACGCTTCAGGCTNNNNTNN
NNTNNNNGAYGACCACGTTCCCATCT.

In an alternative embodiment, the 3′ primer anneals to at least a portion of the constant region of IgD and comprises the sequence:

(SEQ ID NO: 8)
TGTCCAGCACGCTTCAGGCTNNNNTNNN
NTNNNNGGGCTGTTATCCTTTGGGTG.

In an alternative embodiment, the 3′ primer anneals to at least a portion of the constant region of IgE and comprises the sequence:

(SEQ ID NO: 9)
TGTCCAGCACGCTTCAGGCTNNNNTNN
NNTNNNNAGAGTCACGGAGGTGGCATT.

In an alternative embodiment, the 3′ primer anneals to at least a portion of the constant region of IgG (IgG1, IgG2, IgG3 and IgG4) and comprises the sequence:

(SEQ ID NO: 10)
TGTCCAGCACGCTTCAGGCTNNNNTNN
NNTNNNNAGTAGTCCTTGACCAGGCAG.

In an alternative embodiment, the 3′ primer anneals to at least a portion of the constant region of IgM and comprises the sequence:

(SEQ ID NO: 11)
TGTCCAGCACGCTTCAGGCTNNNNTNN
NNTNNNNTCGTATCCGACGGGGAATTC.

References to the term “anneal” as used herein, refer to the process of complementary sequences of single-stranded DNA or RNA pairing by hydrogen bonds to form a double-stranded polynucleotide. The term is often used to describe the binding of a DNA probe, or the binding of a primer to a DNA strand during a polymerase chain reaction.

In one embodiment, when the second nucleic acid is as defined in step (b) (i), the kit additionally comprises a primer that anneals to a polyA tail.

In one embodiment, the non-annealing nucleic acid sequence is a universal sequence which may be recognised by a universal 3′ primer. Examples of universal 3′ primers include, but are not limited to, M13 Reverse (−27), M13 Reverse (−48), SP6, T3, T7 EEV, T7 Reverse, T7 Term, pBluescript KS, pBluescript SK, 3′pGEX, 5′pGEX, GST-Tag, pTrcHis-Reverse, CMV-Reverse, pBAD Reverse, pTRE 3′, pTRE 5′, RVprimer3, Rvprimer4, GLprimer 1, GLprimer 2, SV40-Promoter, U6 Primer and EBV-Rev primer. Therefore, in one embodiment, the universal sequence is one which anneals to a universal 3′ primer comprising the sequence: TGTCCAGCACGCTTCAGGC (SEQ ID NO: 12). In a further embodiment, the universal sequence is one which anneals to the universal 3′ primer sequence: GATACGGCGACCAATGT (SEQ ID NO: 13). Therefore, in a further embodiment, the third nucleic acid sequence is a universal 3′ primer.

In one embodiment, the two or more second nucleic acid sequences each additionally comprises a detectable label. Examples of a detectable label include but are not limited to a protein and/or sequence tags. Therefore, in a further embodiment, the detectable label is an RNA barcode. The term “RNA barcode” as used herein refers to random sequences of nucleic acids which are part of a primer sequence used to uniquely tag each RNA, cDNA or DNA molecule prior to library amplification or sequencing. These can be incorporated during the reverse transcription step and/or during the PCR steps. Advantageously, molecular barcoding allows for correction of PCR and sequencing errors and improves the quantitative potential of immune repertoire analysis.

In one embodiment, the kit comprises two or more, three or more, four or more, five or more or six or more second nucleic acid sequences. In a further embodiment, the kit comprises six second nucleic acid sequences.

In one embodiment, the secondary nucleic acid sequence(s) comprise sequences selected from: GGCCTCAGTGAAGGTCTCCTGCAAG (SEQ ID NO: 14); GTCTGGTCCTACGCTGGTGAAACCC (SEQ ID NO: 15); CTGGGGGGTCCCTGAGACTCTCCTG (SEQ ID NO: 16); CTTCGGAGACCCTGTCCCTCACCTG (SEQ ID NO: 17); CGGGGAGTCTCTGAAGATCTCCTGT (SEQ ID NO: 18); and TCGCAGACCCTCTCACTCACCTGTG (SEQ ID NO: 19).

The kit as described herein has particular application in multiplex amplification reactions, such as polymerase chain reaction. In one embodiment, said kit additionally comprises a polymerase, nucleotide triphosphates, a polymerisation buffer and/or water. Alternatively, the kit as described herein may also have application in a reverse transcription reaction. Therefore, in an alternative embodiment, said kit additionally comprises a reverse transcriptase, a reverse transcription buffer, nucleotide triphosphates, dithiothreitol (DTT) and/or water. Alternatively, the kit as described herein may also have application in both a reverse transcription and polymerase chain reaction. Therefore, in an alternative embodiment, the kit additionally comprises, a polymerase, nucleotide triphosphates, a polymerisation buffer, a reverse transcriptase, a reverse transcription buffer, dithiothreitol (DTT) and/or water. In a further embodiment, the kit additionally comprises instructions to use said kit in accordance with the methods described herein.

In one embodiment, the nucleic acid sequences are DNA.

Method

According to a second aspect of the invention, there is provided a method for amplifying immunoglobulin sequences comprising performing an amplification reaction on cDNA from a biological sample obtained from a human or animal subject, using the kit as defined herein to amplify the immunoglobulin sequences between the first and third nucleic acid sequences.

The protocol presented herein is the first methodology for parallel capture of variable gene diversity together with Ig class and subclass composition of B-cell repertoires in a single reaction. The ability to detect all Ig classes/subclasses simultaneously allows reconstruction of the complete trajectory of clonal evolution to an antigen from a single sample time point without the need for cell separation based on isotype expression.

It will be appreciated that complementary DNA (cDNA) may be generated by reverse transcription from an RNA template. Therefore, selection of suitable reagents, selected from a list comprising: a reverse transcriptase; a reverse transcription buffer; nucleotide triphosphates; dithiothreitol (DTT); and water, will be known to one skilled in the art.

Additional optional steps, such as cDNA clean up, and the benefits thereof will also be known to one skilled in the art and included accordingly. Examples of cDNA clean-up methods include, but are not limited to: phenol extraction; and use of commercial purification kits and reagents, such as spin-column based nucleic acid purification and bead based nucleic acid purification, in particular use of solid phase reversible immobilization beads or columns such as AMP XP beads or NucleoSpin PCR Clean-up, or extraction of product after agarose gel electrophoresis.

It will be known to one skilled in the art that an amplification reaction is a process to amplify nucleic acid. Examples of amplification reactions include, but are not limited to: polymerise chain reaction; loop-mediated isothermal amplification; nucleic acid sequence based amplification; strand displacement amplification; and multiple displacement amplification. Selection of suitable reagents, selected from the list comprising: a polymerase; nucleotide triphosphates; a polymerisation buffer; and water, will be known to one skilled in the art.

The reverse transcription and amplification may be combined in reverse transcription-polymerase chain reaction (RT-PCR). Examples of RT-PCR include, but are not limited: to one-step RT-PCR; and two-step RT-PCR, nested RT-PCR with more than one PCR steps. It would be known to one skilled in the art the necessary requirement of each of these RT-PCR methods. In one embodiment, the RT-PCR is one-step RT-PCR. In an alternative embodiment, the RT-PCR is two-step RT-PCR or a one or two-step RT-PCR followed by additional PCR amplification (nested).

Quantification of the immunoglobulin sequences may also be desired, therefore, in one embodiment, the method as defined herein, comprises quantification of the immunoglobulin sequences. Examples of quantification methods include but are not limited to use of end-point RT-PCR (relative RT-PCR, competitive RT-PCT, comparative RT-PCR) or real-time RT-PCR (SYBR Green, TaqMan Probes, Molecular Beacon Probes, Scorpion Probes, Multiplex Probes).

Sequencing and Computational Analysis

According to a third aspect of the invention, there is provided a method for characterisation of a B-cell repertoire comprising the method for amplifying immunoglobulin sequences as defined in herein, additionally comprising the steps of:
(a) sequencing the amplified product as defined herein to generate sequencing data; and
(b) computational analysis of the sequencing data in step (a) to characterise the B-cell repertoire.

The parallel capture of variable gene diversity together with Ig class and subclass composition of B-cell repertoires in a single reaction extends the practical applications of immune repertoire sequencing, and allows for detailed characterisation of the structure and function of B-cell populations in health thus facilitating the detection of specific immune perturbations in disease. This enables the genetic monitoring of B-cell maturation from a naïve to an antigen experienced state and the relationship between antibody specificity and effector functions.

References to the term “B-cell repertoire” as used herein, refer to the different immunoglobulins produced by the immune system.

References to the term “sequencing” as used herein, include any method or technology that is used to determine the order of nucleotides in a nucleic acid. Examples of sequencing include, but are not limited to, first generation sequencing (e.g. Sanger sequencing and Gilbert sequencing) and second or next-generation sequencing (e.g. Illumina sequencing).

In one embodiment, the sequencing data represents the genetic material from a single cell or multiple cells. In a further embodiment, the sequencing data represents the genetic material from a single cell. In an alternative embodiment, the sequencing data represents the genetic material from multiple cells.

In one embodiment, the computational analysis comprises one or more methods selected from: trimming of the primer sequence(s) used to reverse transcribe; trimming of the primer sequence(s) used to amplify the corresponding RNA transcript; and trimming of the untranslated regions of the represented RNA transcript. It will be known by one skilled in the art when use of one or more of these methods is necessary and when best to incorporate said methods, if any, into the work flow of computational analysis.

In one embodiment, the computational analysis of step (b) comprises the steps of:
(i) identification of constant regions of the immunoglobulin sequences present in the amplified product.

In a further embodiment, the computational analysis of step (b) comprises the steps of:
(i) identification of constant regions, or a subset thereof, of the immunoglobulin sequences present in the amplified product.

In a further embodiment, identification of constant regions of the immunoglobulin sequences present in the amplified product makes use of a reference gene database. In a yet further embodiment, identification of constant regions, or a subset thereof, of the immunoglobulin sequences present in the amplified product makes use of a reference gene database. In still a further embodiment, identification of constant regions of the immunoglobulin sequences present in the amplified product makes use of a reference gene database for each gene region containing at least one isotype region. In a still yet further embodiment, identification of constant regions, or a subset thereof, of the immunoglobulin sequences present in the amplified product makes use of a reference gene database for each gene region containing at least one isotype region. Such methods include, but are not limited to: methods of assigning isotype usage of a sequence with exact or partial homology from a reference gene database; methods of assigning regions of a sequence pertaining to the variable region (the region encoded by the IgV to the IgJ) and extraction of genetic information relating to the sequence region downstream of the IgJ segment (more distal than the IgV). It will be known that assignment to reference IgV and IgJ genes may include an exact or partial identity to a reference gene database.

In one embodiment, the computational analysis defined herein uses k-mer matching, where k=10 and with a minimum of 5 exact k-mer matches within the constant region for acceptable identity. In a further embodiment, the identity is determined by the region with highly k-mer score. It will be known to one skilled in the art that different parameters or measures of homology to the reference is possible, and can be highly dependent on alignment or homology method and/or whether gaps are permissible.

In one embodiment, the computational analysis as defined herein, additionally comprises:
(ii) trimming the constant regions identified in step (i) to include variable regions of the immunoglobulin sequences.

In a further embodiment, identifying the variable region within the DNA sequence makes use of a reference gene database. Such methods include, but are not limited to: methods of assigning the region of the sequence corresponding to the constant region by exact or partial homology to a reference gene database, thus inferring the region encoded by the IgV to the IgJ; and methods of assigning regions of a sequence pertaining to the variable region (the region encoded by the IgV to the IgJ). It will be known that assignment to reference IgV and IgJ genes may include an exact or partial homology to a reference gene database.

In one embodiment, the computational analysis as defined herein, additionally comprises:
(iii) joint analysis of the variable regions and the constant regions.

In a further embodiment, the computational analysis as defined herein, additionally comprises:
(iii) joint analysis of the variable regions and the constant regions, or a subset thereof.

In a further embodiment, the joint analysis of the variable regions and the constant regions uses the linked constant region usage information. In a yet further embodiment, the joint analysis of the variable regions and the constant regions, or a subset thereof, uses the linked constant region usage information. Such methods include, but are not limited to: defining subsets of sequences in the resulting sequence repertoire based completely or in part on constant region usage, wherein said subsets include, but are not limited to: BCR sequences associated with single and/or multiple isotypes; BCRs associated with single and/or multiple isotypes and/or additional sequencing information such as BCR mutational status. For example, the computational analysis defined herein may be employed in defining a subset of sequences based on BCRs associated with IgM and/or IgD that are unmutated and which represent primarily BCRs produced by naïve B-cells. Alternatively, the collection of BCRs associated with IgA1-2, IgE and/or IgG1-4 represent BCRs from class-switched B-cells and can be analysed collectively.

Further applications of the computational analysis defined herein include, but are not limited to: analysis of differences in V, D, and/or J gene usages; analysis of mutational profiles; analysis of differences in nucleotide or amino usages, features and properties; and analysis of differences in repertoire structure between subsets of sequences (e.g. measurements of clonality). For example, the computational analysis defined herein may be employed in analysis of the differences in CDR3 region lengths or differences in the number of negatively charged amino acid residues in the CDR3 region that have the propensity to bind to negatively charge antigen, such as DNA.

Further applications of the computational analysis defined herein include analysis of similarities and relationships of variable regions of sequences defined by isotype class and/or subclass usage, wherein said analysis uses methods including, but not limited to: studying co-expression between isotype classes and/or subclasses, or groups of BCRs based completely or in part on constant region usage; and studying co-evolution between subsets of sequences based completely or in part on constant region usage for phylogenetic methods, network analysis, nucleotide or amino usage analysis.

Further applications of the computational analysis defined herein include the joint analysis of the variable region of BCR together with the isotype usage associated with single cells, where a single cell may be associated with one or more isotype class, wherein said analysis includes, but is not limited to: analysis of the relationships between variable regions derived from individual cells associated with one or more isotype class; and analysis of subsets of cells defined based completely or in part on constant region usage.

It will be appreciated that the biological sample may be any mammalian derived, non-mammalian derived or synthetic biological sample. In one embodiment, the biological sample is mammalian derived. In a further embodiment, the biological sample is from a list including but not limited to: human, mouse, macaque, llama, fish, rat, bird, cow, ferret and rabbit. In a further embodiment, the biological sample is selected from a list including but not limited to: whole blood; dried blood spot; organ tissue; sputum; faeces; saliva; sweat; plasma; and serum.

According to a further aspect of the invention, there is provided a method of computational analysis of the constant and variable regions of an immune receptor, comprising the steps of:
(i) identification of one of said regions of the immune receptor;
(ii) trimming the region identified in step (i) to include the other region of the immune receptor not identified in step (i);
(iii) joint analysis of both of the regions.

In the context of infectious diseases, isotype restriction of variable gene usage can determine the establishment of specific antigen-specific responses, important for the successful resolution of infection and generation of long-term immunity. Therefore, in one embodiment, said immune receptor is a B-cell receptor or T-cell receptor.

It will be appreciated that the method of this aspect of the invention can also include the amplifying and/or sequencing of genetic material encoding for the full length or partial length of any antigen binding region with a mixture of two or more constant regions. Thus, in one embodiment, the method may include any one or more of the following options:
(a) where the antigen binding region include gene fragments encoded by a T-cell receptor V or J genes (within 70% amino acid similarity); and/or
(b) where the antigen binding region may include gene fragments encoded by a non-B-cell receptor V or J genes (less than 70% amino acid similarity from natural hosts); and/or
(c) where the antigen binding region and constant region are derived from the same species (defined by within 70% amino acid similarity from the genome of a host species); and/or
(d) where the antigen binding region and constant region sequences originate from different species, strains, or synthetically designed (not based on immunoglobulin or T-cell receptor constant regions (defined as within 70% sequence similarity from a species), but derived from other regions of a genome or on a synthetically designed gene fragment); and/or
(e) where the antigen binding region and/or constant region sequences may be variants of those found in any species, or a combination of species; and/or
(f) where the antigen binding region is comprised of the rearrangement of multiple gene fragments plus a “constant” region, defined as a region that does not directly participate in antigen binding.

In a further embodiment, options (a) to (f) may be generated from a combinatorial library or e.g. phage display.

Uses

In a further aspect of the invention, there is provided the use of the kit and/or method as defined herein in a screening method for the identification of therapeutic antibodies and/or vaccines.

In a further aspect of the invention, there is provided the use of a kit and/or method as defined herein, in a screening method for monitoring of disease progression and responses to therapy in B-cell malignancies.

In one embodiment, said disease is selected from an autoimmune disease, an allergic disease, an infectious disease, an immunodeficiency, a lymphoproliferative disorder or a cancer.

The benefits presented by the complete isotype characterisation of B-cell repertoires can contribute to more accurate diagnosis and understanding of immune-mediated diseases where class and/or subclass focusing of immune responses is often associated with distinct patterns of disease progression. Furthermore, IsoTyper can readily be used for monitoring the B-cell malignancies over the course of disease or over a particular treatment regimen, where the reproducibility of the assay is of major importance. Detection of underlying class-switching and evolution of leukemic clone demonstrates an important utility of IsoTyper for early detection of residual disease or recurrence post therapy. Therefore, application in screening methods in the fields of vaccinology and immunology, such as immunogenetics and immune-oncology, in particular in the monitoring of CLL, is encompassed by the invention.

Improved characterisation of B-cell responses would also support prophylactic and therapeutic intervention. For example, analysis and precise information on the characteristic of a protective immune response against a specific disease can serve as a template to drive vaccine discovery and development. Information on the specificity of such antibodies can help identify vulnerable epitopes on a pathogen while the class and subclass of such antibodies inform on desired effector functions such as engagement of Fc receptors at the surface of immune cells, recruitment of the complement system, activity at mucosal surface or antibody stability. Deeper analysis of naturally occurring antibody response in individuals who control a specific infection can therefore inform the rational design of vaccine antigen and vaccine delivery.

Determination of sequence information of antibodies with a desired effector functions can also support the development of biological material that can serve in a therapeutic setting to control or clear an ongoing infection as well as in preventative action via passive immunisation. For example, a first in man study is ongoing exploring safety and efficacy of an anti-HIV-1 broadly neutralizing antibody in controlling HIV viremia in infected individuals (Caskey et al. (2015) Nature 522, 487-491). Both prophylactic as well as therapeutic approaches are needed in the control of existing and emerging infectious diseases such as HIV, influenza or haemorrhagic fevers.

According to a further aspect of the invention, there is provided a method for monitoring an autoimmune disease, an allergic disease, an infectious disease, an immunodeficiency, a lymphoproliferative disorder, a cancer, or a vaccinal response of an individual comprising any of the following steps:
(a) usage of two or isotypes within related sequences, sharing >85% V-D-J sequence identity;
(b) the pattern of hypermutation of related sequences sharing >85% V-D-J sequence identity between two or more isotypes;
(c) the V, D and/or J gene usage of related sequences sharing >85% V-D-J sequence identity between two or more isotypes;
(d) the relationship between two or isotypes and two or more full length or partial V-D-J sequences; and/or
(e) monitoring of antigen-specific responses mediated by two or more isotypes in infection, vaccination, immune-mediated disease based on known antigen-specific sequence.

The following studies and protocols illustrate embodiments of the methods described herein:

Materials and Methods

Reverse Transcription

Prepare a mix of the Constant region specific 3′ primers with 10 μM final concentration of each of the primers in the mixture. Make RT-PCR Mix 1 (see below) adding the template RNA last.

RT-PCR - mix I

| Reagent (Mix I): | Volume (μL) per reaction |
|---|---|
| Reverse gene-specific primer mix | 1 |
| 10 mM dNTP Mix | 1 |
| Template RNA (up to 500 ng)* | a |
| Nuclease-free H₂O | b |
| Total volume | 14 |

*RNA concentration might vary depending on sample availability.

The range 50 ng-300 ng RNA is optimal, but the minimum input is 5 ng. More than 500 ng input RNA is suboptimal and reduces the specificity of the PCR. RNA should be extracted from biological samples in an RNAse-free environment and preferably on ice to reduce RNA degradation. Any RNA extraction method which allows for removal of genomic DNA and produces high quality RNA (as tested by BioAnalyser) can be used.

This protocol was optimised for use with RNeasy Micro or Mini Kit Plus (Qiagen) depending on the starting cell number. RNA should be stored at −80° C. and repeated freeze/thaw cycles should be avoided as they can affect RNA quality. Heat the RT-PCR mix with the template RNA to 65° C. for 5 minutes and immediately incubate on ice for at least 1 minute. Centrifuge briefly and add 6 μL of RT-PCR mix II:

RT-PCR - mix II

| Reagent: | Volume (μL) per reaction |
|---|---|
| 5X First-Strand Buffer | 4 |
| 1 μl 0.1M DTT | 1 |
| 1 μl SuperScript III RT | 1 |
| Total | 6 |

Incubate at 50° C. for 60 min followed by 70° C. for 15 min. Freeze the cDNA products at −20° C. or proceed immediately to cDNA clean-up step (proceeding to clean-up immediately is recommended to avoid freeze/thaw cycles of the cDNA).

cDNA Clean Up

This step is beneficial when RNA barcodes are incorporated in the primers used during the reverse transcription. The protocol was optimised for use with AMX XP beads (Beckman Coulter) but alternative column or bead-based methods can also be used.

Vortex AMP XP beads. Add 36 μL per 20 μL reaction (or corrected amount—1.8× times the cDNA reaction volume) of beads to cDNA and pipette mix 10 times. Incubate for 8 minutes at RT. Place plate/tubes on the Magnet plate. Wait for 2 minutes. Aspirate the cleared solution from the reaction plate and discard. Take plate from the magnet and spin down. Place it in the Magnet. Aspirate and discard flow through. Add 30 μL of H₂O. Pipette up and down 10 times. Place plate on the magnet. Wait for 2 minutes. Take the cDNA. cDNA can be stored at −20° C. but for best results proceed straight to PCR.

PCR with HiFI qPCR KAPA Biosystems (#KK2702)

Prepare a mix of the Variable region specific 5′ primers with 10 μM final concentration of each of the primers in the mixture. Prepare the following PCR MasterMix for a 50 μL reaction.

PCR MasterMix (50 μL reaction)

| Reagent: | Volume (μL) |
|---|---|
| 2x KAPA buffer | 25 |
| 3'universl primer (10 μM) | 1 |
| V gene primer mix (10 μM, each) | 1 |
| H₂O | 11 |

Add 12 μL of the clean cDNA to the PCR Master Mix and briefly spin the plate down. Incubate under the following thermal cycling condition:

PCR Thermal cycling program:

| | |
|---|---|
| 1 cycle: | 95° C. 5 min |
| 5 cycles: | 98° C. 5 sec |
| | 72° C. 2 min |
| 5 cycles: | 98° C. 5 sec |
| | 65° C. 10 sec |
| | 72° C. 2 min |
| 30 cycles: | 98° C. 20 sec |
| | 60° C. 1 min |
| | 72° C. 2 min |
| Final extension: | 72° C. 7 min |

Take 5-10 μL of the PCR product and run on an agarose gel to determine success of amplification. Expected PCR product is around 500 bp.

### Example 1: Multiplex PCR with Reverse Primer Barcoding is the Optimal Strategy for Accurate Capture of BCR Repertoires

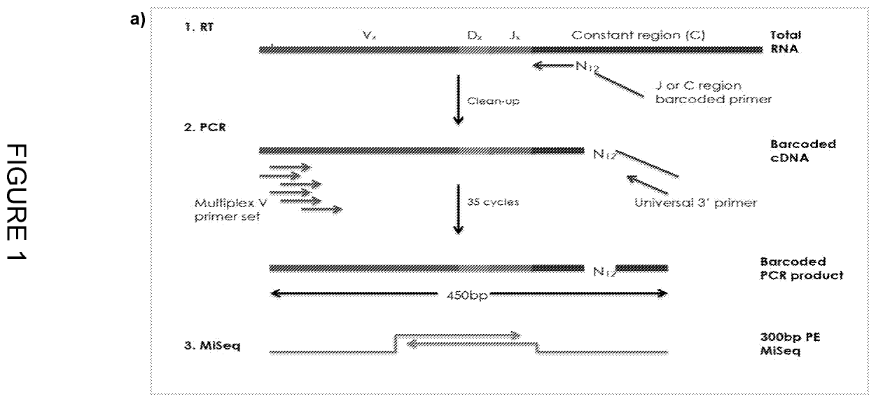

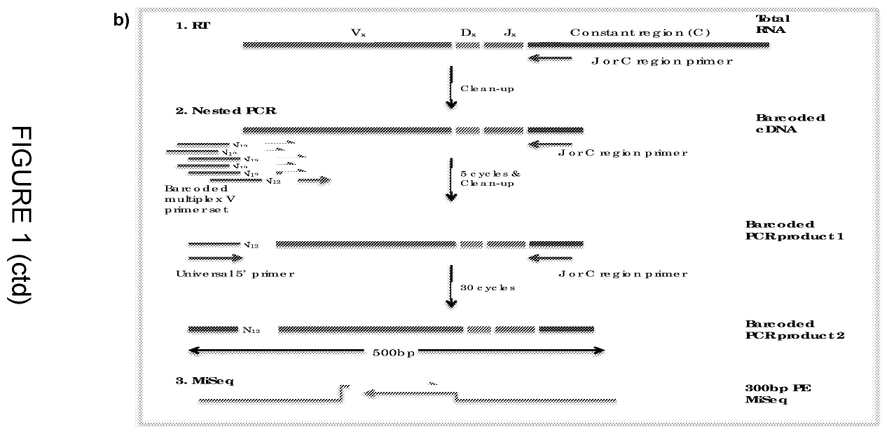

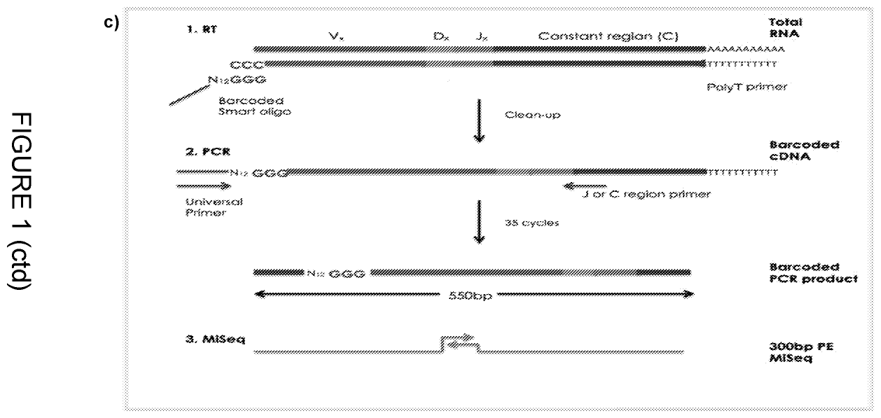

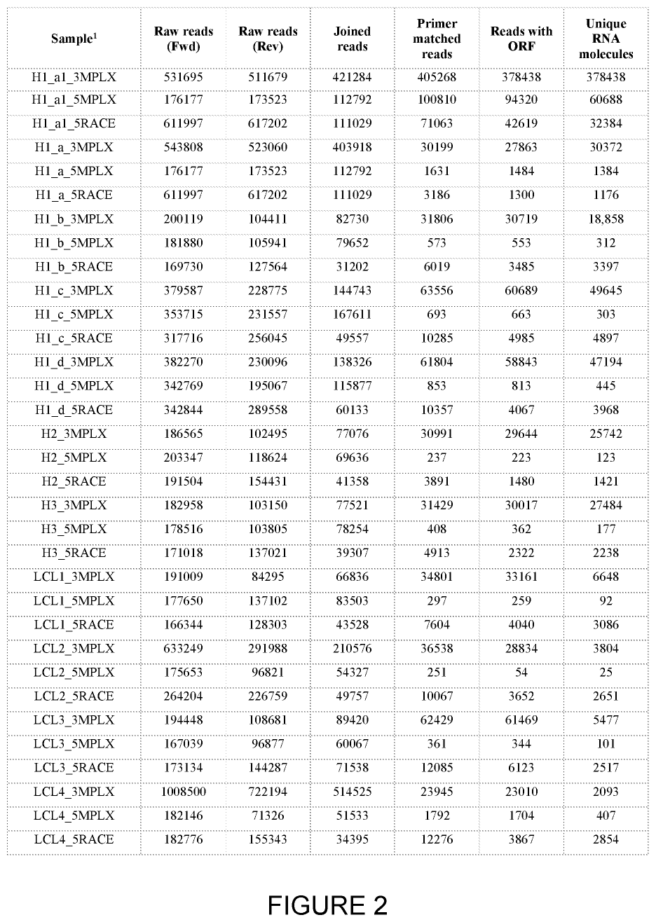

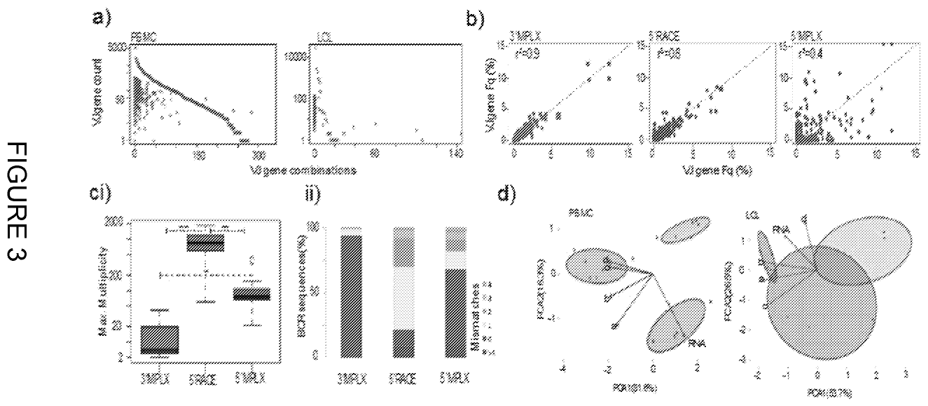

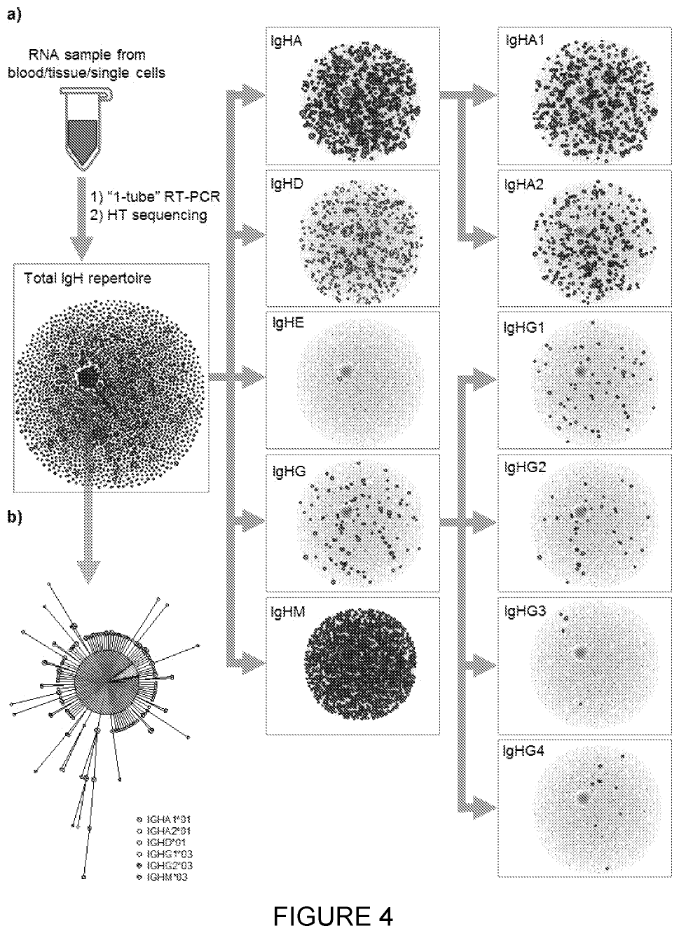

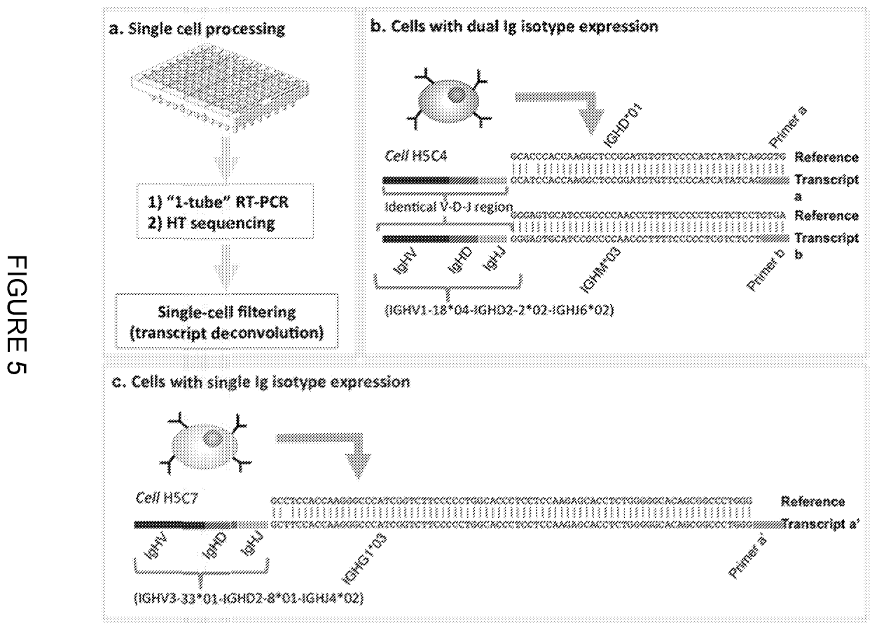

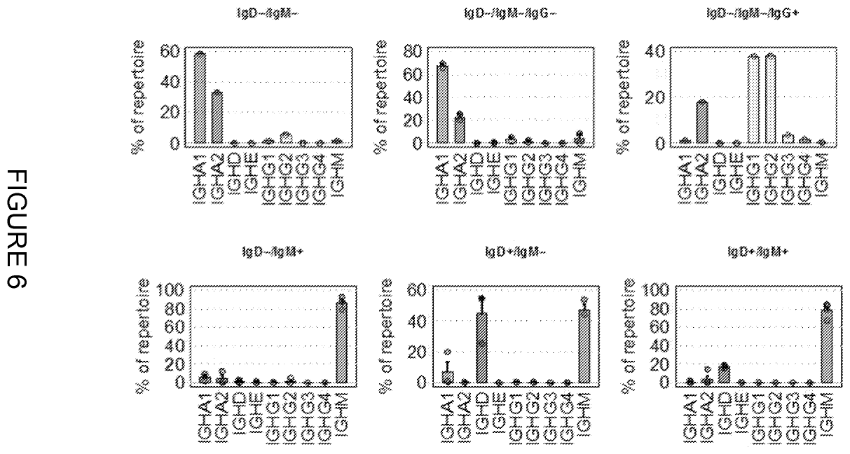

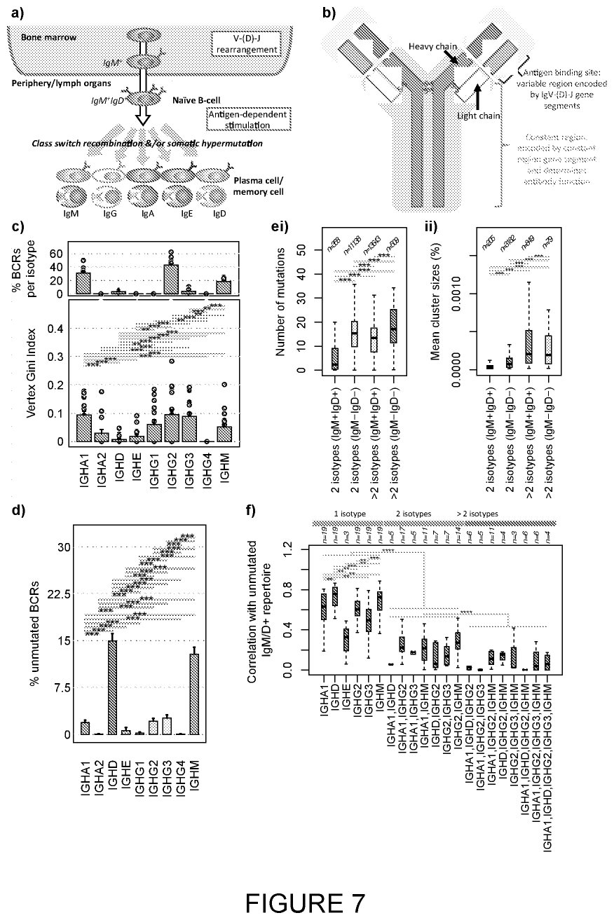

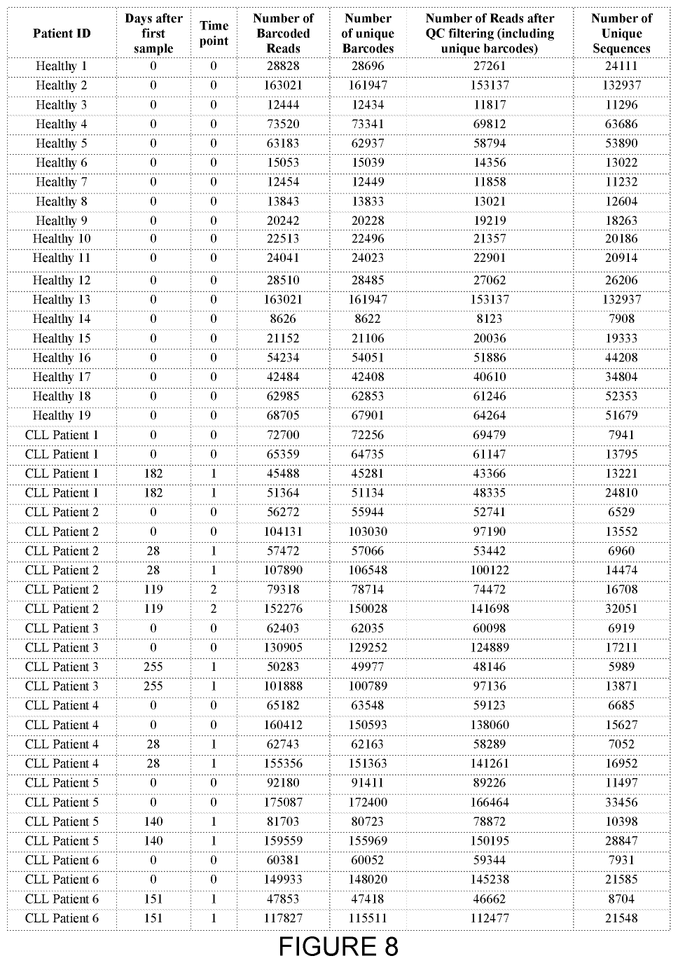

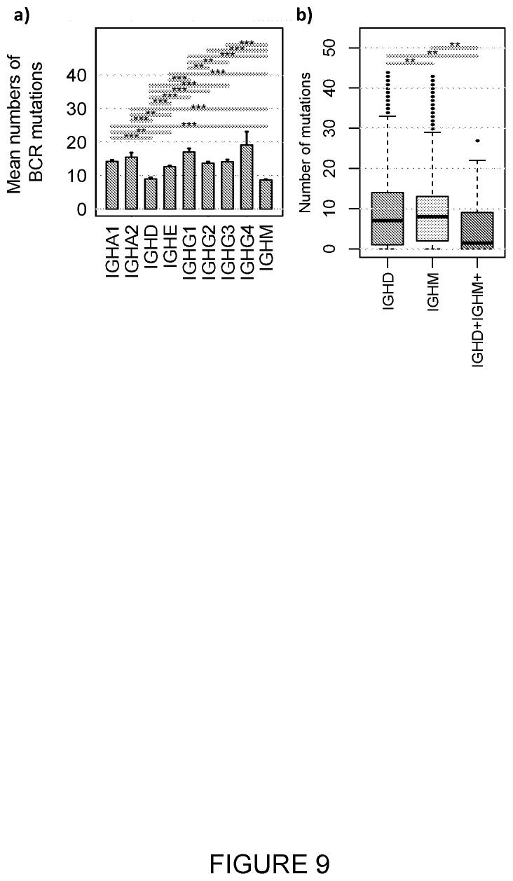

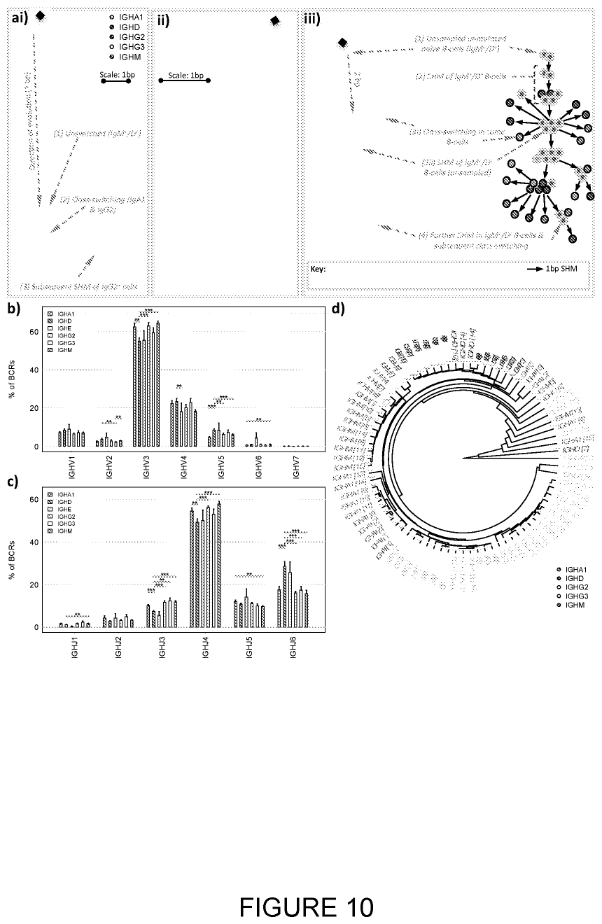

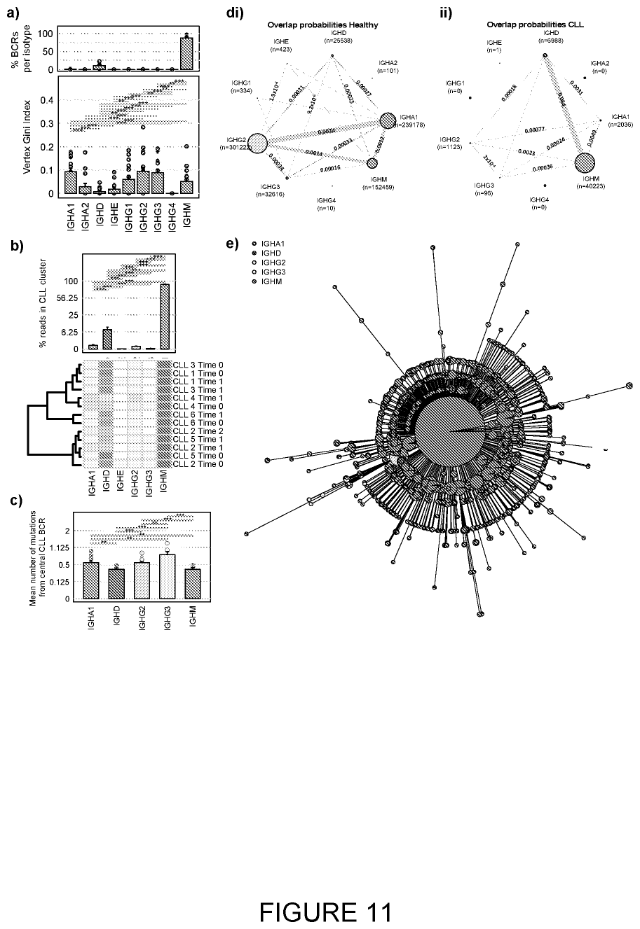

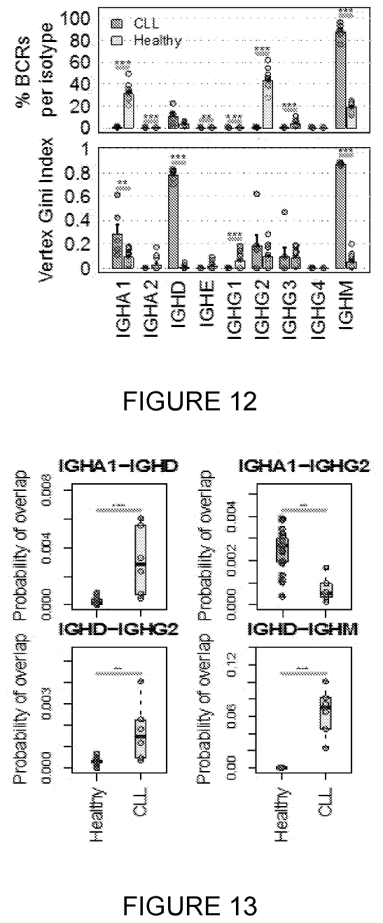

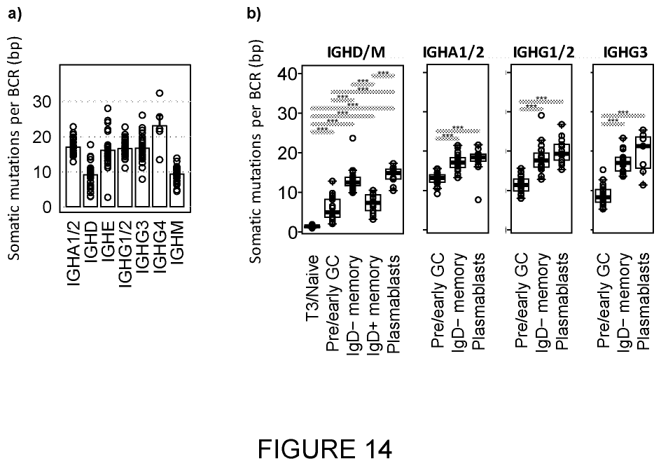

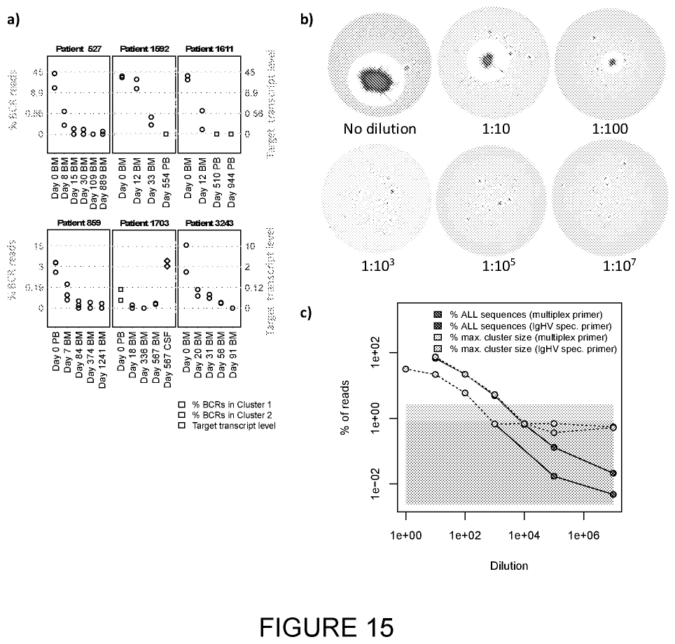

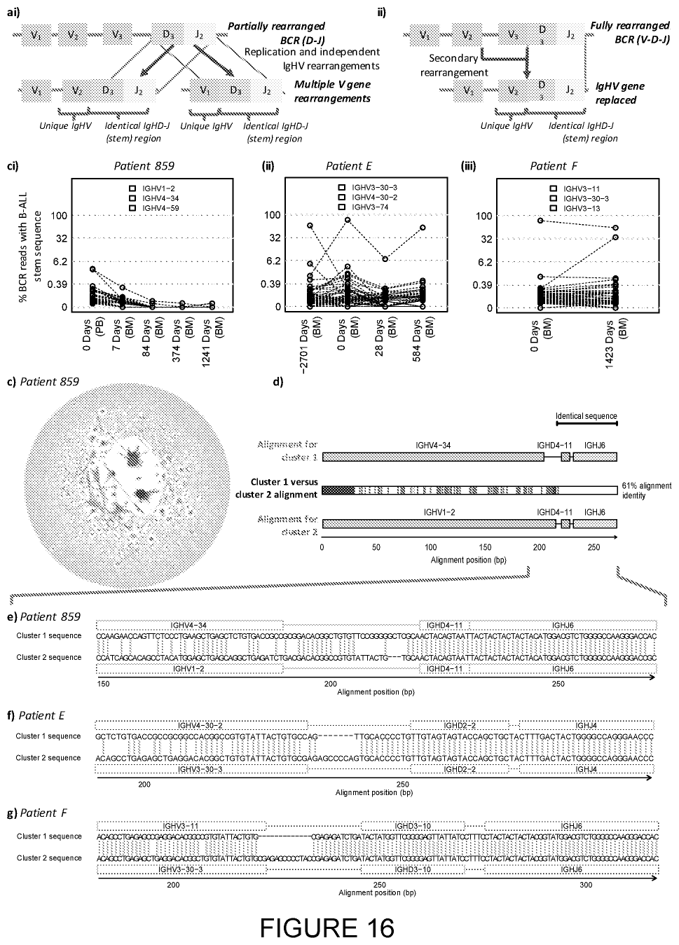

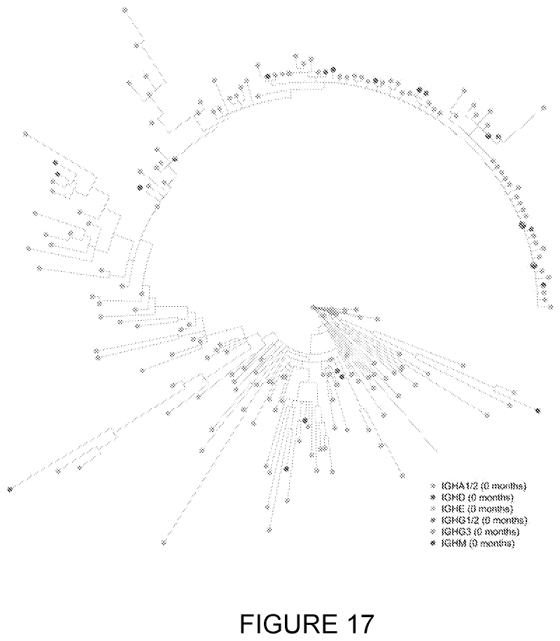

Capturing accurately the full genetic complexity of immune receptor repertoire by high-throughput sequencing poses substantial technical challenges, including PCR and sequencing error, skewed transcript amplification and insufficient transcript or gene capture efficiency during nucleic acid amplification. To ensure the accurate representation of B-cell receptor repertoires, the inventors compared three methods of amplification and molecular barcoding (FIG. 1) across a range of peripheral blood mononuclear cells (PBMCs) and lymphoblastoid cell line (LCL) samples (FIG. 2). The inventors assessed the sensitivity and reproducibility of repertoire capture as well as the barcode profiles characteristic of each method (FIG. 3 a-c). Principle component analysis of the derived network parameters showed repertoire clustering by barcoding method with total number of sampled BCR molecules being the main parameter explaining the observed variance (FIG. 3 d), Furthermore, each amplification strategy showed substantial differences in the degree of introduced amplification bias with 3′MPLX method capturing the most BCR diversity with least amplification bias. On the basis of these results, multiplex BCR amplification with primer barcoding during reverse transcription (3′MPLX) was shown to be the most efficient at capturing immune repertoire diversity capturing between 9-90× more unique RNA molecules, with increased sensitivity of transcript recapture for low frequency BCRs. Therefore, the inventors adopted the 3′MPLX barcoding for the basis of a pan-isotype BCR amplification strategy.

### Example 2: IsoTyper Protocol is Based on 3′MPLX Molecular Barcoding and Enables Pan-Isotype BCR Profiling of Bulk and Singe-Cell Populations

The inventors developed a 3′MPLX barcoded primer set for parallel amplification of all immunoglobulin classes and subclasses in a single PCR reaction. This enabled capture of both IgH VDJ and constant region genes providing high-resolution repertoire characterization from a single RNA sample. Using IsoTyper, the inventors computationally extracted individual IgA, IgD, IgE, IgG and IgM repertoires from the sequencing data, identified the contribution of separate Ig subclasses (IgA1-2 and IgG1-4) to the total repertoire and resolved the combined isotype distribution in the context of each single VDJ clone (FIG. 4). In addition to sequencing of bulk cell populations, the inventors applied IsoTyper for the immune repertoire analysis of flow-sorted CD19⁺CD20⁺CD5⁺ peripheral blood single B-cells, a population enriched in dual-positive IgM⁺IgD⁺ B-cells (FIG. 5 a). Indeed, the inventors identify both cells expressing single isotype (IgG1, FIG. 5 b), and cells, co-expressing IgM and IgD with identical variable V-D-J gene regions (FIG. 5 c). To ensure accurate distinction between isotype-specific populations, CD19⁺CD27⁺ B-cells were sorted into six different populations based on the expression of IgD, IgM, IgG surface markers. Using the complete isotype-specific primer set for each cell population, it was possible to resolve accurately the Ig class composition expected by the surface expression profile of the respective population (FIG. 6). IgD⁺/IgM⁻ cells showed high RNA expression of IgM RNA, despite the low surface abundance of IgM BCR. This likely represents the expression of IgM/IgD isotypes on a single transcript and further processing via alternative splicing. These demonstrated the utility of IsoTyper for accurate isotype decomposition of B-cell populations from both bulk-cell and single-cells samples.

### Example 3: Isotype-Specific Lymphocyte Populations Vary in Size and Diversity in Healthy B-Cell Repertoires

The total diversity of the expressed BCR repertoire reflects the overall lymphocyte composition and the varying degrees of clonal evolution of distinct cell subsets, associated with their function and activation state. IsoTyper enables quantitation of isotype-specific B-cell subsets, as well as assessment of their stage of clonal evolution by characterisation of variable gene diversity (FIG. 7 a-b). To demonstrate this, the inventors characterised the BCR repertoires of 19 PBMC samples from healthy individuals and assessed the size and diversity of B-cell populations from each Ig subtype (FIG. 8). As different B-cell subsets have differing numbers of RNA molecules per cell, repertoires were analysed within this context. The healthy repertoires were dominated by IgM, IgA1 and IgG1 subtypes (FIG. 7 c), consistent with previous reports of peripheral blood composition with predominance of naïve B-cells (˜64% of peripheral blood) as well as IgA⁺, IgG⁺ or IgM⁺ B memory cells (˜30% of peripheral blood) (Perez-Andres, M., et al. (2010) Cytometry B Clin Cytom 78(1), S47-60). The high percentages of BCRs of IgA1, IgG1 classes likely represent also plasmablast/plasma cell populations (Mei, H. E., et al. (2009) Blood 113, 2461-2469) which constitute a small proportion of circulating cells (˜2.1% of the peripheral blood (Perez-Andres, M., et al. (2010) supra)) but express high levels of BCR RNA (>1000 fold more per cell than naïve B-cells), thus are enriched in the total RNA B-cell repertoire. Isotype-specific subsets exhibited varying degree of BCR diversity, consistent with their expected function and maturation stage IgD⁺ B-cells were the most diverse lymphocyte population (lowest Vertex Gini Index), and where a high proportion of these are IgD⁺ naïve cells. The highest degree of clonality was observed for IgA1 and IgHG1-3 subsets reflecting the clonal expansion of antigen-experienced and class-switched B-cell subsets with high-abundance of identical BCRs.

### Example 4: IsoTyper Enables Identification of Activated B-Cell Populations and Evolution of Isotype-Specific Responses

The number of mutations within a BCR sequence relates to the degree of affinity maturation undergone by corresponding B-cell clone, which in turn relates to the degree of antigen-exposure and activation experienced by the clone (Weiser, A. A., et al. (2011) Int Immunol 23, 345-356). The inventors used IsoTyper to determine which isotype classes are associated with zero mutations from germline that, by definition, will not have undergone affinity maturation and should be associated with naïve or unmutated antigen-experienced (T-independent) B-cell clones. B-cell populations of IgD and IgM isotypes showed significantly higher percentage of unmutated BCRs (averages of 12.84% and 14.98% respectively) compared to switched IgA1-2 and IgG1-4 populations (averages of 0.053-2.61%) (FIG. 7 d). Unmutated V genes were further enriched in BCRs with dually expressed IgD⁺ IgM⁺ isotypes (49.12%), previously described as a population of naïve mature B-cells (Peterson, D. A., et al. (2007) Cell host & microbe 2, 328-339). The unmutated IgM and IgD V-J gene usage frequencies were highly correlated (p-values<10⁻²° for healthy individuals), further suggestive of a co-evolutionary nature between the two subclasses and defining IgD⁺ IgM⁺ double positive cells as a predominantly naïve B-cell population. The varying degrees of SHM within BCR of each isotype class reflects the stages during affinity maturation at which the B-cells start to express each isotype, where IgHG1 and IgHG4 exhibit the highest mean mutations per BCR (17.042 and 19.167 mutations respectively) (FIG. 9 a) and likely represent class-switch events occurring late in the process of Ab affinity maturation. IgM⁺IgD⁺ dual positive cells show lower rates of mutation compared to single-positive IgM or IgD populations, consistent with the observed high level of unmutated sequences in this population described above (FIG. 9 b).

### Example 5: IsoTyper Reveals a Step-Wise Process of Affinity Maturation and Immune Focusing of the B-Cell Repertoire from Naïve to Antigen Experienced

On the basis that the IgHM⁺IgHD⁺ unmutated pool of BCRs represents primarily the naïve B-cell population, we investigated the role of Ig isotype on the process of affinity maturation and immune-repertoire evolution from naïve to antigen-experienced state. The changes in BCR repertoires during the course of differentiation and class-switching were demonstrated by the significant increase in size and mutation frequency of clones that have undergone class-switching (2 isotypes, IgM⁻IgD⁻ and >2 isotypes) compared to naïve IgHM⁺IgHD⁺ ones (FIG. 7 e). This is consistent with the predominance of naïve B-cells with unmutated BCRs in IgHM⁺IgHD⁺ clones and suggests that such clones represent early stages of an affinity maturation process. This is further demonstrated by correlations of IgHV-J gene usages between the naïve unmutated IgHM⁺IgHD⁺ repertoires and the repertoires of each subtype combination, where a high correlation suggests low deviation from naïve and less immune focusing. The class-switched repertoires showed lower IgHV-J gene usage correlations with the naïve unmutated IgHM⁺IgHD⁺ repertoire, likely an early signature of antigen-driven selection away from germline variable gene usage (FIG. 7 f). Distinct differences between the IgHG subclasses V-J gene usages reflect the different nature of the IgHG subclass responses, for example, IgHG3 had significantly lower correlations with the naïve repertoire compared to IgHG2. Interestingly, greatest degree of immune-repertoire focusing was observed for BCRs associated with multiple subtypes (p-values<0.0005, FIG. 7 f). Clones of >2 isotypes that are also IgHM⁺IgHD⁺ are significantly more mutated than IgHM⁺IgHD⁺ clusters that have not class-switched (mean mutations 5.205 versus 12.965 respectively (FIG. 7 e). Loss of IgD and IgM expression in clones of >2 isotypes (i.e. IgD⁻ IgM⁻ clones) resulted in the highest level of somatic mutation, suggesting a stepwise trajectory of mutations and class-switching away from IgHM. Clones with the expression >2 isotypes are significantly larger than those with only 2 isotypes (FIG. 7 eii) in accordance with the likely late stage of antigen driven evolution and clonal expansion. This outlines a model of evolution of B-cell diversity towards generation of poly-isotype B-cell response with multiple class-switching events in the context of a single clone (FIG. 10a).

### Example 6: IsoTyper Reveals Class-Specific Antigenic Niches and Isotype-Restriction of Variable Gene Usage

To further characterise the degree of immune-repertoire focusing associated with naïve vs. activated B-cell populations, the inventors compared the frequencies of all V and J genes across the sampled Ig classes (FIG. 10 b, c). IGHV3 and IGHJ4 were the most highly expressed genes across all isotypes (except IgA2, where IGHJ2 was most common) but individual Ig isotypes exhibited significant differences in the frequencies of each variable gene. Hierarchical clustering of VJ gene usage profiles of all healthy repertoires showed significant clustering according to Ig class across all healthy repertoires (FIG. 10 d). This is indicative of isotype restriction of different antigenic binding niches and provides further evidence for the relationship between class-switching and somatic hypermutation during the evolution of antigen-specific B-cell responses.

### Example 7: IsoTyper Reveals Sub-Clonal Diversification and Class-Switching within Leukemic Clones in Chronic Lymphocytic Leukaemia

Having characterised the immune architecture in health, the inventors then explored the distinctive features of class-switching and affinity maturation in the context of disease, namely in chronic lymphocytic leukaemia (CLL). By sequencing of Ig variable genes, PBMCs from CLL patients have been previously shown to exhibit extensive clonal expansion outside of the context of direct antigenic stimulation (Bashford-Rogers, R. J., et al. (2013) Genome research 23, 1874-1884). Here, the inventors show that isotype class usage is significantly different from healthy individuals with significant over-representation of IgHM⁺ and IgHD⁺ isotypes in CLL coupled to significantly lower expression of IgHA1 and IgHG2 (p-value<0.005, FIG. 11 a and FIG. 12). As expected, the majority of BCRs derived from the highly expanded leukemic clusters within each patient are IgHM or IgHD (averages of 89.7% and 9.6% respectively). Interestingly, IgHA1, IgHE, IgHG2 and IgHG3 isotype classes were shown to contribute to the CLL clone, comprising between 0.042-2.72% of the total CLL cluster based on their BCR sequences (FIG. 11 b) demonstrating CLL sub-clonal diversification on both isotype and variable gene level. With AID facilitating and regulating both somatic hypermutation and class-switching (Arakawa, H., et al. (2004) PLoS Biol 2, E179) we investigated whether the two processes appear intrinsically linked in a clone that is not under explicit antigenic selection pressure. Indeed, usage of class-switched isotypes within the CLL clones (IgA1, IgHG2 and IgHG3) is associated with greater numbers of mutations away from the central CLL BCR in all CLL patients sampled. This suggests an ongoing process of SHM and B-cell diversification in the leukemic clone (FIG. 11 c).

### Example 8: Differential Isotype Co-Evolution in Health and Disease

The analysis of healthy repertoires revealed a step-wise process of B-cell activation and diversification of BCRs towards poly-isotype response with immune focusing across several class-switched B-cell populations. To further characterise the degree of isotype co-evolution in response to a given antigen, the inventors estimated the probability that a BCR sequence is shared between any two isotype classes. Conditional overlap probabilities were calculated for every possible isotype pair and accommodated for different numbers of sequences per group. Each individual isotype class co-clustered together across samples (co-clustering p-value<10 Peron, S., et al. (2008) supra). Healthy individuals show significant overlap between BCR repertoires of IgHA and IgHG2 with IgHM, reflecting the populations of memory plasmablasts of these isotypes within the peripheral blood (Weller, S., et al. (2004) Blood 104, 3647-3654), and highlighting further the model of immune-repertoire focusing and establishment of antigenic niche by the activated cell populations. By contrast, the pattern of isotype co-evolution observed in CLL reflects mostly the leukemic clonal state of the B-cell repertoire with significant BCR overlap between IgHM⁺ and IgHD⁺ populations (FIG. 11 d and FIG. 13), where the phylogenetic trees of largest clusters in each sample demonstrates the sharing of the different isotype classes (FIG. 11 e).

### Example 9: IsoTyper Reveals a Step-Wise Process of Affinity Maturation and Immune Focusing of the B-Cell Repertoire from Naïve to Antigen Experienced

The BCRs with that expressed IgD or IgM exhibited the lowest level of somatic hypermutation compared to class-switched BCRs, previously described as a population of naïve mature B-cells (FIG. 14a) (Peterson, D. A., et al. (2007) Cell host & microbe 2, 328-339). This is consistent with the predominance of naïve B-cells with unmutated BCRs in IgHM⁺IgHD⁺ clones and suggests that such clones represent early stages of an affinity maturation process. The varying degrees of SHM within BCR of each isotype class reflects the stages during affinity maturation at which the B-cells start to express each isotype, where IgHG1/2 and IgHG4 exhibit the highest mean mutations per BCR (17.042 and 19.167 mutations respectively) (FIG. 14a) and likely represent class-switch events occurring late in the process of Ab affinity maturation.

SHM levels significantly differ between B-cell populations (FIG. 14b), with lowest SHM in T3/naïve B-cells as expected, and increases from pre/early GC, IgD-memory to plasmablasts for all isotypes. IgD⁺ memory has significantly lower SHM than IgD-memory, reflecting lower mutational propensity in extrafollicular pathways (Berkowska, M. A., et al. (2011) Blood 118(8), 2150-8). Furthermore, SHM levels significantly differ between isotypes, with increased SHM in pre/early GC class-switched BCRs compared to IgHD/M, reflecting the developmental trajectory of B-cell isotype usage.

### Example 10: Pathogenic Clone Tracking Using B-Cell Repertoire Analysis in B-Cell Lymphoblastic Leukaemia

Longitudinal samples from 6 B-cell lymphoblastic leukaemia (B-ALL) patients taken over the course of therapy were analysed for the presence of residual leukaemia by qPCR for transcript levels of fusion genes (treated as per UKALL2003 protocol; Bashford-Rogers, R. J., et al. (2016) Leukemia 30, 2312-2321). Additionally, BCR sequencing was performed on peripheral blood (PB) from 18 healthy individuals aged 20-75 years. After filtering, network analysis (Bashford-Rogers, R. J., et al. (2013) Genome Res. 23(11), 1874-84)[2] was performed on BCR sequencing data verifying clonality in all B-ALL primary diagnostic samples (largest cluster sizes of 5.7-83.64% of the total BCR repertoire) and the day 567 sample from patient 1703 (largest cluster 3.83%). By comparison, the largest clusters from the healthy individuals averaged 0.60% (standard deviation of 0.64%, range 0.14-2.577%). A computational pipeline was developed to identify B-ALL clonotypic BCRs in the diagnostic sample and search diluted or serial patient samples for identical or related BCRs, allowing for a set number of base-pair (bp) mismatches (≤8 bp in this study). Clonotypic sequences were identified (clusters representing ≥2.5% of the entire repertoire, above the 95^{th} percentile of the healthy range) in the primary diagnostic and relapse samples from all 6 patients. BCR sequencing concurred closely with qPCR transcript levels (red/green versus blue lines, FIG. 15a), demonstrating strong correlations between the percentage of clonotypic B-ALL BCRs and qPCR T/C ratios (R²-values>0.87), whilst B-ALL clonotypic BCR sequences were detected in all qPCR positive samples. High reproducibility was observed between the network structures of two independent PCR amplification and sequencing runs.

To quantify the sensitivity of BCR sequencing we performed a titration experiment using serial 10-fold dilutions of a known clonal B-ALL RNA sample (1592_A) into healthy peripheral blood RNA. With 31.41% of all BCR sequences in the undiluted sample related to the leukemic cluster, ALL clonotypic BCRs were detected in dilutions as low as 1 in 10⁷ healthy peripheral blood RNA molecules (FIG. 15b-c).

### Example 11: Pathogenic Clone Tracking Using B-Cell Repertoire Analysis in B-Cell Lymphoblastic Leukaemia Through V-Gene Replacements

B-cell clones may further diversify through the process of V-gene replacements. IgHD-J combinations (including junctional regions), known as “stem sequences” are stable in instances of V-gene replacements, and can be computationally detected associated with different IgVH gene usages in high-throughput sequencing data (FIG. 16a) (Bashford-Rogers, R. J., et al. (2016) Leukemia 30, 2312-2321). By comparing the frequencies of these stem sequences in healthy individuals, we account for false-positive detection rates for each stem sequence (i.e. the chance that the same stem sequence can be generated by chance in independent B-cells). We report that secondary rearrangements are very common in B-ALL, with an average of 32.52 different IgHV genes combined with the stem sequence per B-ALL (range 9-59 IgHV genes: above 99^{th} percentile for healthy individuals). By determining the frequency of each stem sequence in unrelated B-ALL patients, our false detection rate was 9.245×10⁻⁶. Examples of cases where the clones are clearly part of the leukemia were identified in patients 859, E and F, in which large subclones exhibited identical IgHD-J, but different V genes (FIG. 16b-d).

### Example 12: Phylogenetic Analyses Reveals Continued Class-Switching to IgE in Eosinophilic Granulomatosis with Polyangiitis (EGPA) Patients

EGPA is an autoimmune condition that causes inflammation of small and medium-sized blood vessels in patients with a history of airway allergic hypersensitivity, and presenting with elevated serum IgE levels. To assess the role of IgE class-switching, phylogenetic trees of all expanded IgE-associated clones present at diagnosis were generated. Given that each clone is likely to bind a different set of antigen, as expected there is heterogeneity in the phylogenetic tree structures. IgE-associated expanded clones in EGPA were predominantly associated also with multiple other isotypes (demonstrated in the tree in FIG. 17).

### DISCUSSION

Current strategies for immune repertoire sequencing focus largely on Ig variable gene diversity and have provided important insights into sequence determinants of antigen specificity in infection, vaccination and autoimmunity. Understanding variable gene diversity in isolation, however, has limited capacity to uncover the process of B cell maturation in the context of an adaptive immune response which relies on extensive B-T cell interaction resulting in Ig class-switching and further B cell selection and maturation.

The IsoTyper protocol presented here is the first methodology for parallel capture of variable gene diversity together with Ig class and subclass composition of B-cell repertoires in a single reaction. This enables the genetic monitoring of B cell maturation from a naïve to an antigen experienced state and the relationship between antibody specificity and effector functions. The ability to detect all Ig classes/subclasses simultaneously allows reconstruction of the complete trajectory of clonal evolution to an antigen from a single sample time point without the need for cell separation based on isotype expression. This extends the practical applications of immune repertoire sequencing and allows for detailed characterisation of the structure and function of B-cell populations in health, thus facilitating the detection of specific immune perturbations in disease.

In the context of infectious diseases, isotype restriction of variable gene usage can lead to the establishment of isotype-specific response to an antigen and determine the success of pathogen neutralisation and generation of long-term immunity. This is of particular importance for vaccine design where the distinct Ab effector profiles characteristic of Ig isotype classes and subclasses can affect the efficacy of a vaccine. This is demonstrated in a HIV vaccine trial where a protective immune response is only present after generation of IgG3, but not IgG4 Abs and is independent of T cell cytotoxicity or Ab neutralisation properties (Chung, A. W., et al. (2014) Science Translational Medicine 6 (228), 228-238). Co-evolution of IgG3 and IgG1 Abs to identical antigenic epitope as part of successful vaccine-induced protection in the same study further demonstrates the need for simultaneous monitoring of the compete isotype composition of a response to an antigen to ensure accurate assessment of B cell evolution.

The generation of broadly neutralising Ab responses is a strategy exploited by most recent vaccine design efforts, but is often restricted by the low abundance of such antibody classes and thus leads to limited long-term protection. As an example, the majority of anti-HIV bnAbs isolated from infected individuals (VRC-like antibodies) are IgG1 isotype and use members of VH1 gene family. However, the analysis presented herein of healthy repertoires VH1 genes shows the lowest frequency of expression in a IgG1 context compared to all other Ig classes, suggestive of particular immune selection against this variable gene-isotype combination. Such selective pressure can also affect any vaccine-induced or therapeutic bnAbs and thus limit the natural response to HIV or the success of anti-HIV therapy. Therefore, IsoTyper-enabled monitoring of the relationship between SHM (antigen adaptation) and class-switching in the context of an antigen-specific immune response can uncover key immune signatures of protection or susceptibility and thus enable the development of vaccines with improved efficacy.

Due to the distinct effector functions associated with each Ig isotype, the complete isotype characterisation of B-cell repertoires can contribute to more accurate diagnosis and understanding of immune-mediated diseases where subclass focusing of immune responses is often associated with distinct patterns of disease progression, as demonstrated in autoimmunity (Verpoort, K. N., et al. (2006) Arthritis Rheum 54(12), 3799-3808), allergies (Bogh, K. L., et al. (2014) Mol Immunol 58(2), 169-176), infectious diseases (Afridi, S., et al. (2012) Malar J 11, 308)

Furthermore, IsoTyper can readily be used for monitoring the B-cell malignancies over the course of disease or over a particular treatment regimen, where the reproducibility of the assay is of major importance. Detection of underlying class-switching and evolution of leukemic clone demonstrates an important utility of IsoTyper for early detection of residual disease or recurrence post therapy.

Together, this shows that IsoTyper is a robust and sensitive strategy for investigation of diverse B cell populations and for qualitative and quantitative characterisation of their Ig class and subclass structure in health, and as a result of immune perturbation in disease and infection.

1. A kit for amplifying immunoglobulin sequences comprising:
(a) two or more first nucleic acid sequences, each of which comprises a 3′ primer which anneals to at least a portion of the constant region of an immunoglobulin class and/or subclass; and
(b) one or more second nucleic acid sequence comprising:
(i) a 5′ primer comprising a sequence which anneals to at least a portion of each immunoglobulin heavy chain variable gene; or
(ii) a 5′ template-switching sequence,
wherein when the second nucleic acid sequence is as defined in (b) (ii), the kit additionally comprises a third nucleic acid sequence which is a 5′ primer corresponding to said template-switching sequence.

2. The kit of claim 1, wherein when the second nucleic acid is as defined in step (b) (i), the kit additionally comprises a primer that anneals to a polyA tail.

3. The kit of claim 1, wherein when the second nucleic acid is as defined in step (b) (i), the two or more first nucleic acid sequences each additionally comprise a detectable label.

4. The kit of claim 3, wherein:
the two or more first nucleic acid sequences each additionally comprise a non-annealing nucleic acid sequence which is identical in each of said two or more first nucleic acid sequences; and
the kit additionally comprises a third nucleic acid sequence complimentary to said non-annealing nucleic acid sequence.

5. The kit of claim 1, wherein the immunoglobulin class is selected from the group consisting of IgA1, IgA2, IgD, IgE, IgG1, IgG2, IgG3, IgG4, IgM, IgK and IgL, IgF, IgT, IgX, IgW, IgY and IgZ IgNAR, the immunoglobulin subclass is selected from the group consisting of IgA1, IgA2, IgD, IgE, IgG1, IgG2, IgG3, IgG4, IgM, IgK and IgL, IgF, IgT, IgX, IgW, IgY and IgZ IgNAR, or both the immunoglobulin class and subclass are selected from the group consisting of IgA1, IgA2, IgD, IgE, IgG1, IgG2, IgG3, IgG4, IgM, IgK and IgL, IgF, IgT, IgX, IgW, IgY and IgZ IgNAR.

6. The kit of claim 1, wherein the immunoglobulin class is selected from the group consisting of IgA1, IgA2, IgD, IgE, IgG1, IgG2, IgG3, IgG4 and IgM, the immunoglobulin subclass is selected from the group consisting of IgA1, IgA2, IgD, IgE, IgG1, IgG2, IgG3, IgG4 and IgM, or both the immunoglobulin class and subclass are selected from the group consisting of IgA1, IgA2, IgD, IgE, IgG1, IgG2, IgG3, IgG4 and IgM.

7. The kit of claim 1, which comprises three or more, four or more, or five or more first nucleic acid sequences.

8. The kit of claim 1 which comprises two or more, three or more, four or more, five or more, or six or more second nucleic acid sequences.

9. The kit of claim 1, wherein the nucleic acid sequences are DNA.

10. A method for amplifying immunoglobulin sequences, comprising:
performing an amplification reaction on cDNA from a biological sample obtained from a human or animal subject, and
using the kit of claim 1 to amplify the immunoglobulin sequences between the first and second nucleic acid sequences.

11. A method for characterization of a B-cell repertoire, comprising:
performing the method for amplifying immunoglobulin sequences of claim 10 to provide an amplified product;
sequencing the amplified product to generate sequencing data; and
conducting a-computational analysis of the sequencing data to characterize the B-cell repertoire.

12. The method of claim 11, wherein the computational analysis of step (b) comprises:
(i) identifying constant regions, or a subset thereof, of the immunoglobulin sequences present in the amplified product.

13. The method of claim 12, additionally comprising:
(ii) trimming the constant regions identified in step (i) to include variable regions of the immunoglobulin sequences.

14. The method of claim 13, further comprising:
(iii) joint analysis of the variable regions and the constant regions, or a subset thereof.

15. The method of claim 10, further comprising quantification of the immunoglobulin sequences.

16. The method of claim 10, wherein the biological sample is mammalian derived.

17. The method of claim 16, wherein the biological sample is selected from the group consisting of whole blood, a dried blood spot, organ tissue, sputum, feces, saliva, sweat, plasma, and serum.

18. A method for identifying a therapeutic antibody or a vaccine, comprising: providing the kit of claim 1.

19. A method for monitoring disease progression and responses to therapy in B-cell malignancies, comprising: providing the kit of claim 1.

20. The method of claim 19, wherein said disease is selected from an autoimmune disease, an allergic disease, an infectious disease, an immunodeficiency, a lymphoproliferative disorder or a cancer.

21. A method for monitoring an autoimmune disease, an allergic disease, an infectious disease, an immunodeficiency, a lymphoproliferative disorder, a cancer, or a vaccinal response of an individual, comprising one or more of (a)-(e):
(a) usage of two or isotypes within related sequences, sharing >85% V-D-J sequence identity;
(b) the pattern of hypermutation of related sequences sharing >85% V-D-J sequence identity between two or more isotypes;
(c) the V, D and/or J gene usage of related sequences sharing >85% V-D-J sequence identity between two or more isotypes;
(d) the relationship between two or isotypes and two or more full length or partial V-D-J sequences; and
(e) monitoring of antigen-specific responses mediated by two or more isotypes in infection, vaccination, immune-mediated disease based on known antigen-specific sequence.

22. A method of computational analysis of the constant and variable regions of an immune receptor, comprising the steps of:
(i) identifying one of a constant region or a variable region of an immune receptor;
(ii) trimming the region identified in step (i) to include the other region of the immune receptor not identified in step (i); and
(iii) performing a joint analysis of both of the regions.

23. The method of claim 22, wherein said immune receptor is a B-cell receptor or T-cell receptor.


SEQUENCE LISTING (19 sequences)

SEQ ID NO: 1 - DNA, 15 bp, Artificial
  Synthetic Oligonucleotide
  misc_feature (1)..(4): n is a, c, g, or t
  misc_feature (6)..(9): n is a, c, g, or t
  misc_feature (11)..(14): n is a, c, g, or t
nnnntnnnntnnnnt

SEQ ID NO: 2 - DNA, 19 bp, Artificial
  Synthetic Oligonucleotide
  misc_feature (3)..(3): y is c or t
gaygaccacgttcccatct

SEQ ID NO: 3 - DNA, 20 bp, Artificial
  Synthetic Oligonucleotide
tcgtatccgacggggaattc

SEQ ID NO: 4 - DNA, 20 bp, Artificial
  Synthetic Oligonucleotide
gggctgttatcctttgggtg

SEQ ID NO: 5 - DNA, 20 bp, Artificial
  Synthetic Oligonucleotide
agagtcacggaggtggcatt

SEQ ID NO: 6 - DNA, 20 bp, Artificial
  Synthetic Oligonucleotide
agtagtccttgaccaggcag

SEQ ID NO: 7 - DNA, 53 bp, Artificial
  Synthetic Oligonucleotide
  misc_feature (21)..(24): n is a, c, g, or t
  misc_feature (26)..(29): n is a, c, g, or t
  misc_feature (31)..(34): n is a, c, g, or t
  misc_feature (37)..(37): y is c or t
tgtccagcacgcttcaggctnnnntnnnntnnnngaygaccacgttcccatct

SEQ ID NO: 8 - DNA, 54 bp, Artificial
  Synthetic Oligonucleotide
  misc_feature (21)..(24): n is a, c, g, or t
  misc_feature (26)..(29): n is a, c, g, or t
  misc_feature (31)..(34): n is a, c, g, or t
tgtccagcacgcttcaggctnnnntnnnntnnnngggctgttatcctttgggtg

SEQ ID NO: 9 - DNA, 54 bp, Artificial
  Synthetic Oligonucleotide
  misc_feature (21)..(24): n is a, c, g, or t
  misc_feature (26)..(29): n is a, c, g, or t
  misc_feature (31)..(34): n is a, c, g, or t
tgtccagcacgcttcaggctnnnntnnnntnnnnagagtcacggaggtggcatt

SEQ ID NO: 10 - DNA, 54 bp, Artificial
  Synthetic Oligonucleotide
  misc_feature (21)..(24): n is a, c, g, or t
  misc_feature (26)..(29): n is a, c, g, or t
  misc_feature (31)..(34): n is a, c, g, or t
tgtccagcacgcttcaggctnnnntnnnntnnnnagtagtccttgaccaggcag

SEQ ID NO: 11 - DNA, 54 bp, Artificial
  Synthetic Oligonucleotide
  misc_feature (21)..(24): n is a, c, g, or t
  misc_feature (26)..(29): n is a, c, g, or t
  misc_feature (31)..(34): n is a, c, g, or t
tgtccagcacgcttcaggctnnnntnnnntnnnntcgtatccgacggggaattc

SEQ ID NO: 12 - DNA, 19 bp, Artificial
  Synthetic Oligonucleotide
tgtccagcacgcttcaggc

SEQ ID NO: 13 - DNA, 17 bp, Artificial
  Synthetic Oligonucleotide
gatacggcgaccaatgt

SEQ ID NO: 14 - DNA, 25 bp, Artificial
  Synthetic Oligonucleotide
ggcctcagtgaaggtctcctgcaag

SEQ ID NO: 15 - DNA, 25 bp, Artificial
  Synthetic Oligonucleotide
gtctggtcctacgctggtgaaaccc

SEQ ID NO: 16 - DNA, 25 bp, Artificial
  Synthetic Oligonucleotide
ctggggggtccctgagactctcctg

SEQ ID NO: 17 - DNA, 25 bp, Artificial
  Synthetic Oligonucleotide
cttcggagaccctgtccctcacctg

SEQ ID NO: 18 - DNA, 25 bp, Artificial
  Synthetic Oligonucleotide
cggggagtctctgaagatctcctgt

SEQ ID NO: 19 - DNA, 25 bp, Artificial
  Synthetic Oligonucleotide
tcgcagaccctctcactcacctgtg